(19) United States
(12) Patent Application Publication
(10) Pub. No.: US 2003/0161866 A1
(43) Pub. Date: Aug. 28, 2003

(54) Novel pharmaceutical formulation to limit or delay surface absorption

(76) Inventors: Paul Kostyniak, Amherst, NY (US); Ross Giese, Elma, NY (US)

Correspondence Address: Michael L. Goldman, NIXON PEABODY LLP, Clinton Square, P.O. Box 31051, Rochester, NY, 14603, US

(21) Appl. No.: 09/982,821

(22) Filed: Oct. 18, 2001

Related U.S. Application Data
(60) Provisional application No. 60/241,398, filed on Oct. 18, 2000.

(51) Int. Cl. A61K 7/42; A61K 31/14

(52) U.S. Cl. 424/449; 514/642

(57) ABSTRACT

The present invention relates to a complex including colloid particles having an ion exchange capacity and having attached one or more ligands with a charge and a hydrophobic domain, and one or more pharmaceutical compounds associated with the colloid particles and/or the one or more ligands, as well as a method of making the complex. The present invention also relates to methods for preventing or reducing surface absorption or for delayed percutaneous delivery of one or more pharmaceutical compounds. The methods involve applying to a surface a complex according to the present invention. Another aspect of the present invention relates to a surface coated with colloid particles having attached one or more ligands with a charge and a hydrophobic domain and one or more pharmaceutical compounds associated with either or both of the colloid particles and the one or more ligands.

This application claims the benefit of U.S. Provisional Patent Application Serial No. 60/241,398, filed Oct. 18, 2000, which is hereby incorporated by reference in its entirety.

### FIELD OF THE INVENTION

This invention relates generally to methods for preventing or reducing surface absorption or for delayed percutaneous delivery of one or more pharmaceutical compounds. In addition, the present invention relates to a complex and a surface including colloid particles having attached one or more ligands and one or more pharmaceutical compounds associated with the colloid particles and/or the one or more ligands, as well as a method of making the complex.

### BACKGROUND OF THE INVENTION

Following the Gulf war, a number of US combatants reported neurological symptoms of unknown etiology (Gulf War Syndrome). Simultaneous exposure to chemicals used has been implicated as a factor in the neurotoxicity (Fradin, “Mosquito and Mosquito Repellents: A Clinician's Guide,” Annals of Internal Medicine, 128: 931-940 (1998); Stenecipher et al., “Percutaneous Permeation of N,N-diethyl-m-toluamide (DEET) From Commercial Mosquito Repellents and the Effect of Solvent,” J. of Toxicology and Environmental Health, 52:119-135 (1997)). The chemicals used included pyridostigmine, permethrine, and DEET (N,N-diethyl-m-toluamide). It is in this light that significant research efforts have been focused not only on determining the cause of the Gulf War Syndrome, but also on improving the safety of insect repellents (Abdou-Donia et al., “Neurotoxicity Resulting from Coexposure to Pyridostigmine Bromide, DEET, and Permethrine: Implications of Gulf War Chemical Exposure,” J. of Toxicology and Environmental Health, 48: 35-56 (1996)). Ideally an insect repellent possesses prolonged repellency towards a selected species of insect without appreciable absorption by the user (Snodgrass et al., Dermal Penetration and Potential for Placental Transfer of the Insect Repellent, N,N-diethyl-m-toluamide,” Am. Ind. Hyg. Assoc., 43: 747-753 (1982)).

DEET is a broad spectrum repellent that is effective against mosquitoes, biting flies, and ticks (Fradin, “Mosquito and Mosquito Repellents: A Clinician's Guide,” Annals of Internal Medicine, 128: 931-940 (1998)). The latest reports show that DEET still remains the standard of currently available insect repellents. It is estimated that more than 38% of the American population uses DEET based repellents, with a world wide use estimated at over 2 million people yearly (Fradin, “Mosquito and Mosquito Repellents: A Clinician's Guide,” Annals of Internal Medicine, 128: 931-940 (1998).

DEET is available in multiple topical formulations including solutions, lotions, creams, gels, aerosols, impregnated towellettes, and pump sprays, with concentrations ranging from 5 to 100%, and varying durations of action. Commercial products in the market are typically formulated in ethyl alcohol or isopropyl alcohol (Stenecipher et al., “Percutaneous Permeation of N,N-diethyl-m-toluamide (DEET) From Commercial Mosquito Repellents and the Effect of Solvent,” J. of Toxicology and Environmental Health, 52:119-135 (1997); Robbins et al., “Review of the Biodistribution and Toxicology of the Insect Repellent N,N-diethyl-m-toluamide (DEET),” J. of Toxicology and Environmental Health, 18:503-525 (1986)).

Even though it has been used for over 40 years, there has been a growing interest in the safety of DEET, and as a result, an increase in the number of absorptive studies done in man and other animals. Several studies have looked at DEET's ability to enhance dermal delivery of certain drugs. These studies suggest that DEET may be a permeation enhancer (Robbins et al., “Review of the Biodistribution and Toxicology of the Insect Repellent N,N-diethyl-m-toluamide (DEET),” J. of Toxicology and Environmental Health, 18:503-525 (1986); Winheuser et al., “The Use of N,N-diethyl-m-toluamide to Enhance Transdermal Delivery of Drugs,” J. of Pharmaceutical Sciences, 71: 1211-1213 (1982); Moody et al., “The Effect of DEET (N,N-diethyl-m-toluamide) on Dermal Persistence and Absorption of the Insecticide Fenitrothion in Rats and Monkeys,” J. of Toxicology and Environmental Health, 22: 471-479 (1987)). Due to its lipophilicity, DEET is prone to absorption through the skin after topical use, especially when high concentrations are used in pediatric populations (Fradin, “Mosquito and Mosquito Repellents: A Clinician's Guide,” Annals of Internal Medicine, 128: 931-940 (1998); Dom, et al., “Insect Repellent Formulations of N,N-diethyl-m-toluamide (DEET) in a Liposphere System,” Journal of American Mosquito Control Association, 11:29-34 (1995); Lipscomb, et al., “Seizure Following Brief Exposure to Insert Repellent N,N-diethyl-m-toluamide,” 21:315-317 (1992); Roland, et al., “Toxic Encephalopathy in a Child after a Brief Exposure to Insect Repellents,” Canadian Medical Association Journal, 132:155-156 (1985), which are hereby incorporated by reference in their entirety). Toxicity of an agent is related to its concentration at the site of action. Therefore the rates of absorption, distribution, biotransformation, and excretion will significantly influence the toxic response exerted by DEET. Variability in absorption among individuals may depend, among other things, on the lipid content of the skin, the size of the dose given, and other drugs that are concomitantly administered along with DEET (Abdou-Donia et al., “Neurotoxicity Resulting from Coexposure to Pyridostigmine Bromide, DEET, and Permethrine: Implications of Gulf War Chemical Exposure,” J. of Toxicology and Environmental Health, 48: 35-56 (1996); Baynes et al., “The Influence of diethyl-m-toluamide (DEET) on the Percutaneous Absorption of Permethrin and Carbaryl,” Toxicology and Applied Pharmacology, 144:332-339 (1997)). Approximately 17% of the dose administered topically is absorbed within two hours of administration. The toxicity of DEET related to this attribute has been a subject of continued research. Several studies have looked into reducing the topical absorption of DEET without compromising the duration of its effectiveness.

Metabolic biotransformation results in detoxification of DEET by cytochrome p450 and plasma hydrolyzing enzymes. DEET is completely metabolized and several metabolites have been characterized. The major metabolites identified as ethyltoluamide and N,N-diethyl m-hydroxymethylbenzamide have been detected using both in vitro and human studies (Selim et al., “Absorption, Metabolism, and Excretion of N,N-diethyl-m-toluamide Following Dermal Application to Human Volunteers,” Fundamentals and Applied Toxicology, 25:95-100 (1995); Moody et al., “Dermal Absorption of the Insect Repellent DEET (N,N-diethyl-m-toluamide) in Rats and Monkeys: Effects of Anatomical Site and Multiple Exposure,” J. of Toxicology and Environmental Health, 26:137-144 (1989); Roland et al. “Toxic Encephalopathy in a Child After a Brief Exposure to Insect Repellents,” Canadian Medical Association Journal, 132:155-156 (1985)). However, when very large doses are given, studies show that a significant portion of the dose is excreted unchanged in the urine (Selim et al., “Absorption, Metabolism, and Excretion of N,N-diethyl-m-toluamide Following Dermal Application to Human Volunteers,” Fundamentals and Applied Toxicology, 25:95-100 (1995); Abdou-Donia et al., “Neurotoxicity Resulting from Coexposure to Pyridostigmine Bromide, DEET, and Permethrine: implications of Gulf War Chemical Exposure,” J. of Toxicology and Environmental Health, 48: 35-56 (1996)).

Urinary excretion accounts for nearly all of the dennally absorbed dose of DEET. It is excreted as metabolites, mainly as conjugates. Almost 90% of the absorbed dose can be recovered in the urine 24 hours after administration of the last dose.

Toxicity reports of great concern in the general population involve encephalopathy, over 90% of which were in children younger than 8 years (Fradin, “Mosquito and Mosquito Repellents: A Clinician's Guide,” Annals of Internal Medicine, 128: 931-940 (1998)). Along with the neurological effects seen with the Gulf War Syndrome, this makes the central nervous system (CNS) the major site of toxicity (Wright et al., “Reproductive and Developmental Toxicity of N,N-diethyl-m-toluamide in Rats,” Fundamentals of Applied Toxicology, 19:33-42 (1992)). Ingestion of large doses of DEET can produce seizures and coma by direct action on the CNS (Heick et al., “Insect Repellent N,N-diethyl-m-toluamide, Effect on Ammonia Metabolism,” Pediatrics, 82:373-376 (1988); Tenebein, “Severe Toxic Reactions and Death Following the Ingestion of diethyltoluamide Containing Insect Repellents,” JAMA, 258:1509-1511 (1987)). DEET crosses the blood brain barrier, and simultaneous exposure with agents that inhibit its metabolism will indirectly increase concentrations that will ultimately cross the blood brain barrier. This has been offered as a likely explanation for the adverse interaction when DEET is used simultaneously with pyridostigmine and permethrine (Abdou-Donia et al., “Neurotoxicity Resulting from Coexposure to Pyridostigmine Bromide, DEET, and Permethrine: Implications of Gulf War Chemical Exposure,” J. of Toxicology and Environmental Health, 48: 35-56 (1996)).

Studies of the pharmacokinetics of DEET, and comparisons of its transdermal absorption in different formulations have been carried out (Qui et al., “Solid Phase Extraction and Liquid Chromatographic Quantitation of Insect Repellent N,N-diethyl-m-toluamide in Plasma,” J. Pharm. Biomed. Anal., 15: 241-250 (1996); Qiu et al., “Pharmacokinetics of Insect Repellent N,N-diethyl-m-toluamide in Beagle Dogs Following Intravenous and Topical Routes of Administration,” J. of Pharmaceutical Sciences, 86:514-516 (1997)).

However, there continues to be a need for an improved formulation of DEET having reduced absorption through the skin, and/or extended duration of protection.

### SUMMARY OF THE INVENTION

The present invention relates to a complex including colloid particles having an ion exchange capacity and having attached one or more ligands with a charge and a hydrophobic domain, and one or more pharmaceutical compounds associated with either or both of the colloid particles and the one or more ligands.

Another aspect of the present invention relates to a method for preventing or reducing surface absorption of one or more pharmaceutical compounds. The method involves applying to the surface a complex including colloid particles having an ion exchange capacity and having attached one or more ligands with a charge and a hydrophobic domain, and one or more pharmaceutical compounds associated with either or both of the colloid particles and the one or more ligands.

Yet another aspect of the present invention relates to a method for delayed percutaneous delivery of one or more pharmaceutical compounds. The method involves applying topically a complex including colloid particles having an ion exchange capacity and having attached one or more ligands with a charge and a hydrophobic domain, and one or more pharmaceutical compounds associated with either or both of the colloid particles and the one or more ligands.

The present invention also relates to a surface coated with colloid particles having attached one or more ligands with a charge and a hydrophobic domain, and one or more pharmaceutical compounds associated with either or both of the colloid particles and the one or more ligands.

Another aspect of the present invention relates to a method of making a complex. The method involves providing colloid particles having an ion exchange capacity and having attached one or more ligands with a charge and a hydrophobic domain, and exposing the colloid particles to one or more pharmaceutical compounds under conditions effective to produce a complex including the one or more pharmaceutical compounds associated with either or both of the colloid particles and the one or more ligands.

The methods, complexes, and surfaces of the present invention offer advantages not obtainable in the prior art. Because the pharmaceutical compounds are associated with the colloid particles and/or the one or more ligands of the present invention, the ability of the pharmaceutical compounds to leach out is reduced. Thus, the pharmaceutical compounds retain their pharmaceutical properties, and the need for repeated coatings to a surface (e.g., the skin) is eliminated or reduced. In addition, because the pharmaceutical compounds are associated with colloid particles having attached one or more ligands, which are inert, they are less likely to be absorbed by a surface (e.g., the skin) and, therefore, they are less toxic to that surface.

### BRIEF DESCRIPTION OF THE DRAWINGS

FIGS. 1 A-B show the chemical structures for N,N-diethyl-m-toluamide (DEET) (FIG. 1A) and hexadecyltrimethylammonium (HDTMA) (FIG. 1B). See Newger, Organic Chemical Drugs and Their Synonyms, 7^{th} Edition, Volumes I-II (1994), which is hereby incorporated by reference in its entirety.

FIG. 2 shows evaporation profiles of IPA from the matrix formulation over time. DEET was dissolved in varying amounts of IPA ranging from 0 ml, 0.5 ml, 1 ml, 1.5 ml, to 2 ml. All samples contained 1g of DEET. Samples 2 to 6 contained 500 mg 1×HDTMA while no clay was added to sample 1. For the formulation with no IPA, weight loss is expressed relative to the amount of DEET contained formulation.

FIG. 3 is a graph showing the effect of aging the formulation on the extent of intercalation. A 2 ml aliquot of spiking solution containing 25 mg of DEET in 30% IPA was intercalated into 250 mg of 1×HDTMA clay. The formulation was allowed to incubate at room temperature for 1, 8, and 30 days. Percent adsorbed is expressed relative to the amount of DEET in the initial spiking solution.

FIGS. 4A-B show the effect of vehicle on the sorption of DEET by HDTMA treated clays. Samples containing 250 mg of clay were incubated with 2 ml of spiking solution for 7 days, centrifuged, then analyzed for DEET in the supernatant. Spiking solutions consisted of varying concentrations of DEET in 30, 40, or 50% IPA. FIG. 4A shows DEET in 1×HDTMA clay. FIG. 4B shows DEET in 3×HDTMA clay.

FIGS. 5A-B show the influence of the HDTMA load on the adsorptive capacity of the clays for DEET. All samples contained 250 mg of the respective clay, and various amounts of DEET dissolved in 2 ml of 30% IPA. FIG. 5A shows the extent of sorption relative to the actual amount of DEET in the spiking solution. FIG. 5B shows the amount of DEET sorbed as a function of equilibrium concentration.

FIG. 6 is a Scatchard plot of the amount of DEET bound by the clay in relation to the fraction of bound:free DEET. Plots compare 0.5×, 1×, and 3×HDTMA treated clay. All samples contained 250 mg of the respective clays, and various amounts of DEET dissolved in 2 ml of 30% IPA.

FIGS. 7A-B show the DEET release kinetics across a dialysis membrane into 30 ml of 30% IPA bathing solution. 2 ml of spiking solution containing 25 mg of DEET in 30% IPA was used to prepare the formulation (DEET only), while the same spiking solution was mixed in with 1×HDTMA treated clay to prepare the sample (DEET+1×). FIG. 7A shows the concentration of DEET in bathing solution versus time. FIG. 7B shows the percent of DEET remaining on the clay relative to time.

FIGS. 8A-B show the DEET release kinetics for a formulation diluted directly into 30 ml of either 30% IPA or 0.9% saline solution. Formulations were prepared by mixing spiking solutions containing either 25 mg (B and C) or 100 mg (E and F) DEET in 30% IPA with 250 mg of 1×HDTMA treated clay. Controls A and D consisted of free DEET spiking solutions, 25 mg or 100 mg, respectively. All samples were centrifuged for 10 minutes prior to collection of supernatant. FIG. 8A shows the concentration of DEET in the bathing solution versus time. FIG. 8B shows plots of percent remaining DEET versus time for A and B only.

FIG. 9 is a graph showing the extent to which either 30% IPA or 0.9% NaCl wash solutions will leach out DEET from a clay matrix and the effect of the volume used. 2 ml of spiking solution of DEET (25 or 50 mg) in 30% IPA was incorporated into 250 mg of 1×HDTMA clay. The formulation was then extracted in incremental volumes of either 30% IPA or 0.9% saline solution.

FIG. 10 is a graph showing the accumulation of DEET in the bathing solution relative to the type and volume of wash solvent. The same samples and procedure were used as in FIG. 8.

FIGS. 11A-C are models for the sorption of HDTMA molecules as a monolayer (FIG. 11A), patchy bilayers (FIG. 11B), and bilayers (FIG. 11C) on smectite clay surfaces. The surfactant head groups are positively charged and balance the charge on the negatively charged clay surfaces. See Li et al., “Counterion Effects on the Sorption of Cationic Surfactant and Chromate on Natural Clinoptilolite,” Environmental Science and Technology, 31:2407-2412 (1997), which is hereby incorporated by reference in its entirety.

### DETAILED DESCRIPTION OF THE INVENTION

The present invention relates to a complex including colloid particles having an ion exchange capacity and having attached one or more ligands with a charge and a hydrophobic domain, and one or more pharmaceutical compounds associated with either or both of the colloid particles and the one or more ligands.

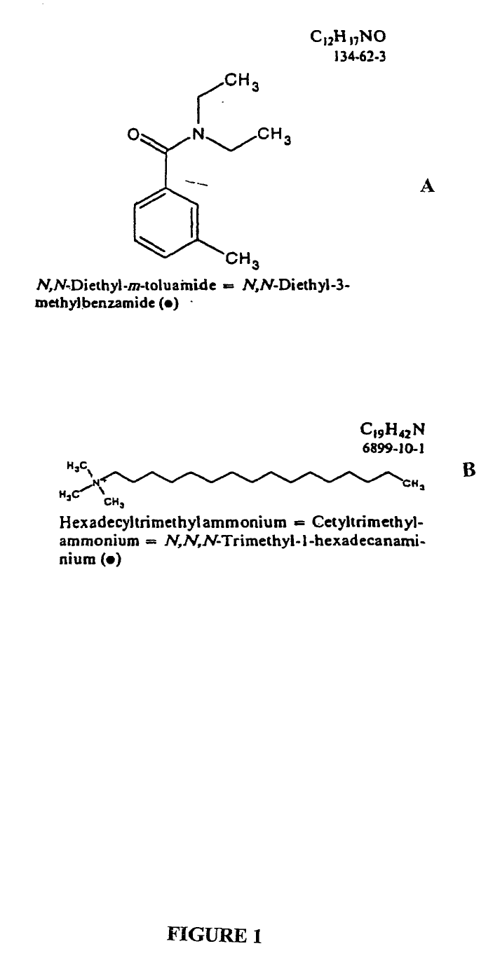

Suitable ligands with a charge and a hydrophobic domain include, but are not limited to, quaternary ammonium compounds, ionic polymers, ionic surfactants, and mixtures thereof. Especially desirable quaternary ammonium compounds include hexadecyltrimethyl ammonium bromide (see FIG. 1B), trimethylphenyl ammonium chloride, and mixtures thereof.

The general formula for the quaternary ammonium compounds (QACs) is as follows:

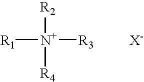

where R₁, R₂, R₃, and R₄ are alkyl groups which may be alike or different. Structurally, these compounds contain four carbon atoms linked directly to one nitrogen atom through covalent bonds. The portion attached to the nitrogen by an electrovalent bond may be any anion, but it is usually chloride or bromide to form the salt. The nitrogen atom with the attached alkyl groups forms the positively charged cation portion. Depending on the nature of the R groups, the anion, and the number of quaternary nitrogen atoms present, the quaternary ammonium compounds may be classified as monoalkyltrimethyl, monoalkyldimethylbenzyl, heteroaromatic, polysubstituted quaternary, bis-quaternary, or polymeric quaternary ammonium compounds.

When QACs or other ligands are used in accordance with the present invention, the complex may also include antimicrobial properties, as described in U.S. Pat. Nos. 6,015,816 and 6,288,076, which are hereby incorporated by reference in their entirety.

In accordance with the present invention, one or more ligands may be attached to the colloid particles. In one embodiment, at least two ligands are attached to the colloid particles. In this embodiment, the at least two ligands are different.

Any inorganic material exhibiting a combination of high surface area and a substantial ion exchange capacity, such as natural and synthetic clay minerals, are useful as colloid particles in the present invention. Preferred inorganic materials have surface areas ranging from 50-1000 m²/g, with surface areas of 500-800 m /g being especially desirable. Useful synthetic types of clay include a synthetic hectorite, which is a layered hydrous magnesium silicate, such as Laponite® (Southern Clay Products, Gonzales, Tex.), a synthetic mica-montmorillonite, such as Barasym® (Baroid Division, NL Industries, Houston, Tex.), and mixtures thereof. Useful natural types of clay include swelling clays such as aliettite, beidellite, nontronite, saponite, sauconite, stevensite, swinefordite, volkonskoite, yakhontovite, hectorite, montmorillonite (such as BP colloid), bentonite, and mixtures thereof. Other useful materials (both synthetic and mineral) include, but are not limited to, zeolites, illite, chlorite, kaolinite, hydrotalcite, talc, halloysite, sepiolite, and palygorskite. Typically, the colloid particles of the present invention have a mean diameter of 1 nm to 1000 microns, with mean diameters of less than 2 microns being preferred.

Clays particularly useful as colloid particles in the present invention are members of the smectite clay mineral group which are distinguished by a large surface area (“S”), the ability to exchange cations, specified by the cation exchange capacity (“CEC”) (which has units of milliequivalents (meq)/100 grams of clay), and by the ability to swell in the presence of water and a variety of organic liquids, specified by the thickness of the clay layers as revealed by X-ray diffraction. The high surface areas of these materials results from three factors: 1) the small particle size which creates a large external surface area, 2) the ability of the clay layers to expand by incorporating between adjacent layers water and various organic liquids which create a large internal surface area and 3) the plate-like morphology of the colloid particles. Smectite clay minerals are layer structures, of the 2:1 type, which have a net negative charge as the result of substitutions of different cations within the individual mineral sheets. The negative charge on the individual layers is balanced by cations, such as sodium, calcium, and magnesium, which are adsorbed onto both the external and internal surfaces of the clay layers. The CEC of smectite clays typically varies between approximately 50 and 150 meq/100 grams.

Examples of specific types of clays from the smectite mineral group include: hectorite (“SHCa−1”, the Source Clay Minerals identification code) (provided by Source Clay Minerals Repository, University of Missouri, Columbia, Mo.) with a CEC 43.9 meq/100 g and S=63.2 m²/g; Cheto montmorillonite (“SAz−1”) with a CEC=120 meq/100 g and S=97.4 m²/g; Washington montmorillonite (“SWa−1”); Wyoming montmorillonite (“SWy−2”) with a CEC=76.4 meq/100 g, S=31.8 m²/g; Laponite® RD with a CEC=73 meq/100 g and S=330 m²/g; and Laponite® RDS with a CEC=73 meq/100 g and S=360 m²/g.

To produce the complex of the present invention, the colloid particles, either free or previously bound to a surface, are subjected to ion exchange reactions whereby one or more ligands with a charge and a hydrophobic domain displace the normal endogenous inorganic cations or anions of the colloid particles. An important feature of this invention is that the modifying ligands are retained by the mineral surfaces even after exhaustive washing.

Specifically, the preparation of the complex is based on the process of ion exchange within the colloid particle. For example, clays have exchangeable cations such as calcium, magnesium, potassium, sodium, and hydrogen on their internal and external surfaces. The cations on the clay mineral surfaces balance the net negative charge that occurs in clay minerals. Two adjacent negatively charged clay layers are held together by the presence of the cations situated between the layers. A typical clay particle can consist of from two to hundreds of such layers, all held together by the electrostatic bonds formed between the cations and the negatively charged surface of the clay layers. This bonding, although strong enough to keep the layers together, is much weaker than the bonding between the atoms that form the layers. This weaker bonding between the layers plus the strong attraction of the interlayer cations for water allows the entrance of water and other molecules into the interlayer space. The endogenous inorganic interlayer cations will be displaced by other inorganic or organic cations contained in the liquid. Thus, if clay particles are suspended in a liquid dispersant containing cations, such as one or more ligands with a charge and a hydrophobic domain, there is an exchange of the endogenous inorganic interlayer cations of the clay for the cations in the liquid dispersant. Accordingly, these cations are referred to as exchangeable cations. An organic cation replaces the inorganic cation in a nearly quantitative manner by this single treatment method. Even though naturally occurring smectites are hydrophilic, addition of organic cations, such as cationic quaternary ammonium compounds (e.g., HDTMA) to replace the inorganic ions occupying the exchange sites will render the clay organophilic. It should also be noted that some colloids have a net positive charge and in such a case, the exchangeable ions would be anions.

In accordance with the present invention, a quantity of ligand in excess of and up to 200% of the CEC can be achieved for loading the colloid particles, as described in U.S. Pat. No. 6,015,816, which is hereby incorporated by reference in its entirety. Although not meaning to be bound by theory, it is believed that this high loading is achieved due to the bonding of the one or more ligands to other ligands. More particularly, in the present invention, all surface and interlayer cations are replaced by one or more ligands in an amount equivalent to 100% of the cation exchange capacity of the colloid particles. Further, other ligands then bond to these ligands, such that a loading of an excess of and up to 200% of the cation exchange capacity of the colloid particles is achieved. The organic cation present in excess of the CEC is probably attached to the organo-clay by interactions between the cation exchanged organic material and an organic salt (U.S. Pat. No. 4,365,030 to Oswald, et al., which is hereby incorporated by reference in its entirety).

To produce the complex, preferably, from 0.1 to 10 wt % of colloid particles, such as clay, is mixed with the liquid dispersant. The liquid dispersant contains sufficient ligand to satisfy fully or partially the ion exchange capacity of the mineral colloid and to form a suspension containing the desired ligand. The liquid dispersant is typically water, but it can be any liquid dispersant. The suspension is then thoroughly mixed and held for up to 24 hours at from 45° to 100° C. The suspension is then centrifuged, decanted, and water washed three times, preferably with distilled water. It is especially desirable to expose the colloid particles to fresh solution three times to ensure maximum loading of the ligand. However, other techniques for producing the complex including colloid particles having attached one or more ligands can be used, such as grinding together the clay and ligand (see, e.g., del Hoyo et al., “Application of phenyl salicylate-sepiolite systems as ultraviolet radiation filters,” Clay Minerals, 33:467-474 (1996), which is hereby incorporated by reference in its entirety) or mixing mechanically the colloid particles and ligand and applying mild heat until the ligand melts into the colloid particles (see, e.g., del Hoyo et al., “Adsorption of Melted Drugs Onto Smectite,” Clay and Clay Minerals, 44:424-428 (1996), which is hereby incorporated by reference in its entirety). The colloid particles may be exposed to different types of ligands in order to bind two or more different types of ligands to the colloid particles. A loading of up to 200% of the cation exchange capacity may be achieved. For instance, if Wyoming montmorillonite (“SWy−2”) is immersed in an aqueous solution of hexadecyltrimethyl ammonium bromide (“HDTMA”), the organic cations of the HDTMA will exchange for the inorganic cations of the clay. In a preferred embodiment, where the ligand is HDTMA, a loading of 1-times the CEC is achieved.

Once exchanged into the clay silicates, the C₁₆ alkylhydrocarbon groups of the HDTMA interact forming an organic partition phase, which is thought to behave like a bulk phase organic solvent, except that now it is fixed on the clay surface and the interlayer (Boyd et al., Layer Charge Characteristics of 2:1 Clay Minerals, CMS Workshop Lectures, Vol. 6, pp. 48-77 (1994), which is hereby incorporated by reference in its entirety).

In accordance with the present invention, one or more pharmaceutical compounds are associated with the colloid particles and/or the one or more ligands. As used herein, associated means associated with the colloid particles and/or the one or more ligands by non-covalent bonding.

Suitable pharmaceutical compounds include, but are not limited to, insect repellents, such as DEET (see FIG. 1A), citronyl, dimethyl phthalate, n-butyl phthalate, 3-benzoyl piperidine, N-toluyl piperidine, and combinations thereof, sunscreens, such as p-aminobenzoic acid, salicylates (e.g., 4-tert-butylphenyl salicylate), butylmethoxydibenzoyl methane, aminobenzoic acid esters, benzophenone cinnamates, and combinations thereof, and combinations of insect repellents and sunscreens. As used herein, the term “pharmaceutical compounds” does not include antimicrobials.

To produce the complex including the one or more pharmaceutical compositions, in a preferred embodiment, from about 1 to about 20 wt % of colloid particles having attached one or more ligands, such as HDTMA modified clay, is mixed with a solution of the pharmaceutical compound, e.g., DEET. The solvent for the pharmaceutical compound solution may be any suitable solvent based on the pharmaceutical compound(s) chosen. For DEET, the solvent is preferably isopropyl alcohol, isopropyl alcohol and water, other alcohols, or a ketone. Suitable concentrations of pharmaceutical compound in the solution depends on the desired use of the resulting complex. However, concentrations of from about 12.5 mg/ml to about 200 mg/ml are desirable. The suspension is then thoroughly mixed and the carrier solvent may then be evaporated. However, other techniques for producing the complex including the one or more pharmaceutical compositions may be used, such as mixing DEET (not in solution) with the colloid particles having attached one or more ligands. The colloid particles having attached one or more ligands may be exposed to different types of pharmaceutical compounds in order to associate two or more different types of pharmaceutical compounds with the colloid particles and/or the one or more ligands. The length of exposure of the colloid particles having attached one or more ligands to the one or more pharmaceutical compounds is determined by the desired quantity of the pharmaceutical compound(s) in the final complex. Preferably, the colloid particles having attached one or more ligands are exposed to the pharmaceutical compound(s) for from about 1 to about 30 days, more preferably, from about 8 to about 30 days.

The sorption of a pharmaceutical compound, such as DEET, into the modified clay may be based on its distribution between the immobilized sorbed organic phase, and the liquid phase. This distribution of DEET in the various phases is related to its ability to partition out of the solvent phase into the organic phase derived from the ligand, e.g., HDTMA.

The complex can be applied to a surface to impart the properties of the pharmaceutical compound to the surface. The complex can be applied to the surface by any means, for example, by spraying, spreading, dipping, or brushing. Preferably, the complex is provided in a topical formulation, such as a solution, lotion, cream, gel, aerosol, impregnated towellette, or pump spray. In this embodiment, the complex is blended with liquids or gels as a suspension to impart the properties of the pharmaceutical compound to the liquid or gel. As discussed above, the complex can be applied in a single step where the colloid particles having attached one or more ligands and one or more pharmaceutical compounds associated with the colloid particles and/or the one or more ligands are applied to the surface. Alternatively, a three-step method can be used, where the colloid particles are applied to the surface, followed by contacting the colloid particles with the one or more ligands, and followed by contacting the colloid particles and/or the one ore more ligands with one or more pharmaceutical compounds. The surface can be any surface to which it is desired to impart the properties of the pharmaceutical compound. Such surfaces include, but are not limited to, skin and cloth. The time of exposure of the surface to the complex can vary from a few seconds to hours, days or longer depending on the application. This process may be repeated to apply more complex to the surface.

For a cloth surface, the thickness of the applied coating can be varied depending upon the need. It is understood that one skilled in the art will be able to select the appropriate thickness for their use.

Once coated onto the surface in question, the pharmaceutical compound(s) in the complex of the present invention does not readily leach out, eliminating the need for repeated coatings. In addition, the complex of the present invention decreases or eliminates absorption of the pharmaceutical compound by the surface, e.g., the skin. Therefore, any toxicity associated with the pharmaceutical compound will be limited or prevented from transferring to the surface on which it is applied. Typical applications include applying the complex to the skin to function as an insect repellent or sunblock. Examples of other surfaces which can be coated with the complex of the present invention include apparel, etc., where the fiber is cotton, natural down, nylon, polyester, rayon or wool, nonwoven disposable diapers, nonwoven polyester, outerwear apparel, polyurethane foam used as a growth medium for crops and plants, pre-moistened towellettes and tissue wipes, tents, tarpaulins, and athletic and casual shoes.

In addition, the complex of the present invention can be applied topically to both natural and synthetic fibers. The fibers that can be used with the complex of the present invention include but are not limited to fibers made of wool, cotton, polyolefin, polyester, polyaramid, cellulose acetate, rayon, nylon, polystyrene, vinyls, acrylics, and polyurethanes.

The complex can be applied to the fiber or fabric by mixing it with a liquid such as water or other solvent or dispersant and then dipping, spraying or washing the fiber or fabric in the mixture. Suitable solvents that can be used in either method to apply the complex include, but are not limited to, aliphatic and aromatic solvents such as alcohols, benzene, toluene, xylene, and hexane. After applying the mixture, the fiber or fabric will be coated with the complex. Therefore, when the pharmaceutical compound is DEET, for mosquitoes, biting flies, or ticks which come into contact with the fiber or fabric, the DEET will inhibit the infestation of the mosquitoes, biting flies, or ticks.

Examples of the type of fiber or fabric products contemplated include, but are not limited to, mattress covers, crib covers, bassinet covers, tents, draw sheets, cubicle curtains, fabric wall covering, fabric base, fabric shower curtains, and clothing such as shirts, socks, shorts, pants, shoes, and the like.

Another aspect of the present invention relates to a method for preventing or reducing surface absorption of one or more pharmaceutical compounds. The method involves applying to the surface a complex including colloid particles having an ion exchange capacity and having attached one or more ligands with a charge and a hydrophobic domain, and one or more pharmaceutical compounds associated with either or both of the colloid particles and the one or more ligands.

In accordance with the method of the present invention, the pharmaceutical compounds are associated with the colloid particles and/or the one or more ligands of the present invention, thus, the ability of the pharmaceutical compounds to leach out is limited. Accordingly, the pharmaceutical compounds retain their pharmaceutical properties, and the need for repeated coatings to a surface (e.g., the skin) is eliminated or reduced.

In addition, because the pharmaceutical compounds are associated with colloid particles having attached one or more ligands, which are inert, they are less likely to be absorbed by the surface on which the complex is applied. Thus, any toxicity associated with the pharmaceutical compound will be less likely to transfer to the surface on which the complex is applied. This is particularly important where the surface is the skin and the pharmaceutical compound is an insect repellent, such as DEET.

As described above, the association of the pharmaceutical compound(s) with the colloid particles and/or ligand(s) decreases the absorption of the pharmaceutical compound(s) through the skin. Thus, the complex of the present invention can be used in a method for delayed delivery of one or more pharmaceutical compounds through the skin.

Accordingly, yet another aspect of the present invention relates to a method for delayed percutaneous delivery of one or more pharmaceutical compounds. The method involves applying topically a complex including colloid particles having an ion exchange capacity and having attached one or more ligands with a charge and a hydrophobic domain, and one or more pharmaceutical compounds associated with either or both of the colloid particles and the one or more ligands.

The present invention also relates to a surface coated with colloid particles having attached one or more ligands with a charge and a hydrophobic domain, and one or more pharmaceutical compounds associated with either or both of the colloid particles and the one or more ligands.

Another aspect of the present invention relates to a method of making a complex. The method involves providing colloid particles having an ion exchange capacity and having attached one or more ligands with a charge and a hydrophobic domain, and exposing the colloid particles to one or more pharmaceutical compounds under conditions effective to produce a complex including the one or more pharmaceutical compounds associated with either or both of the colloid particles and the one or more ligands.

The following examples are presented to further illustrate the invention.

### EXAMPLES

### Example 1—Incorporation of DEET onto Clays

DEET liquid was obtained from Sigma Chemical Co. (St. Louis, Mo.) and it contained 95% N,N-diethyl-m-toluamide, and 5% related isomers. The commercial DEET formulation was Ben's 100 max formula from Tender Corporation (a formulation of 100% DEET). Millipore water was purified in the lab with a conductivity of 1.6 μOhms/cm. Sodium chloride was from Fisher Scientific. The nylon filter membranes for filtering the mobile phase were 0.45 μm and were obtained from Supelco. Hexadecyltrimethylammonium bromide (HDTMA) powder (>99% purity) was obtained from Fisher Scientific. Native BP clay colloid with a Cation Exchange Capacity (CEC) of 0.75 meq./100 g was from Southern Clay Products (Gonzales, Tex.). Clays loaded with HDTMA at 1 and 3 times its CEC (1× and 3×, respectively) were made in the Department of Geology (SUNY Buffalo), while the clay loaded to 0.5 times its CEC (0.5×) was prepared in the Toxicology Research Center. The solvents: isopropyl alcohol (IPA), toluene, and acetonitrile were all HPLC grade. Pre-purified nitrogen gas was used for evaporation of isopropyl alcohol from the samples. The dialysis membrane used had a cut off molecular weight size of 12,000-14,000 and was from Fischer Scientific. The thread used to tie dialysis membranes was non-absorbable surgical silk size 3, from Ethicon. The dialyses membrane tubing was prepared by cutting into 6×9.5 mm long tubes, which were soaked in 30% IPA for three hours prior to filling with the clay/DEET suspension.

Samples were analyzed using a Hewlett Packard 5710 model Gas Chromatograph (GC), a Hewlett Packard 1100 series High Performance Liquid Chromatograph (HPLC), and a Siemens D500 model X-ray diffractometer.

The aims of the preliminary experiments were to: (1) determine if HDTMA modified clays were capable of adsorbing DEET; (2) to find optimal working concentrations for incorporating DEET onto the clay; (3) to identify an effective and suitable solvent for extracting DEET from the clay matrix; and (4) to arrive at a reliable analytical method for quantitating DEET.

In these experiments IPA was only used as a carrier solvent for DEET with subsequent evaporation. Varying amounts of 100% IPA were used in the spiking solution, the aim being to study if the extent of incorporation of DEET was related to the volume of the carrier solvent used.

Spiking solutions of DEET in IPA were prepared with concentrations ranging from 33% to 100% w/v. Into the 7 ml glass vials, 1 g of DEET was weighed, and volumes of IPA ranging from 0 ml to 2 ml of 100% IPA were added. The solution was vortexed for 30 seconds until homogeneity was achieved.

The 1× and 3×HDTMA treated BP clays (500 mg) were added to the individual spiking solutions prepared above. The mixture was vortexed for a minute until all the clay was mixed in with the solution. The consistency of the mixture varied depending on the amount of IPA in the spiking solution. Suspensions with 1 ml or more IPA were highly viscous whereas those with less IPA formed a semi-solid mixture. The samples were then placed in the hood under nitrogen gas to evaporate the carrier solvent, and volatility of DEET and IPA was analyzed using the weight loss profile. Aliquots of these samples were further studied for leachability and analyzed by GC, HPLC, and X-ray diffraction.

The HDTMA clay matrix was then tested for evaporation of DEET/IPA. The main purpose of evaporation process was to remove IPA from the matrix formulation and see if it had affected the incorporation of DEET into the HDTMA treated clay.

Several studies have suggested that evaporation may also affect the dermal persistence of DEET. The aims of this evaporation analysis were to remove the carrier solvent from the formulation and to evaluate the effect of clay on the volatility of DEET.

The samples prepared above were held at room temperature in the hood under nitrogen over a 12 hour period. Evaporation rates were measured every hour.

### Example 2—Leaching of DEET from Clay

To determine the most effective solvent for extracting DEET from the clay mixtures, two solvents were compared. Aliquots of 100 mg each of the samples from above were extracted in triplicate, using 6 ml of either IPA, or toluene, or toluene plus water. The mixtures were vortexed for 1 minute, and then mixed on a rotary mixer for 4 hours until fully suspended. The suspension was then centrifuged at 1800 rpm for 15 minutes, after which the supernatant was analyzed by GC and HPLC. The residue was analyzed by X-ray diffraction in order to determine the basal spacing between clay layers due to the interlayer adsorption of DEET molecules.

### Example 3—Analysis of Samples

Various analytical methods were used to assess the DEET content in both the supernatant and the residue. IPA was evaporated from all samples before extraction with solvent. Evaporation studies were used to assess the effect of IPA on the extent of incorporation of DEET onto the clay. The data was also used to evaluate the influence of HDTMA clay on the volatility of DEET.

Samples of the HDTMA clay/DEET/IPA matrix were mixed as illustrated in Example 1. Volatile components of the matrix were evaporated using a constant flow of nitrogen over 12 hours in fume hood. Control samples of DEET with IPA only and DEET alone were subjected to the same conditions as the DEET with HDTMA treated clay.

Weight loss of the samples over 12 hours was used as a measure of the extent of evaporation. Samples were weighed at the beginning of the experiment (time 0) and at hourly intervals thereafter. Reported values are the difference between the original weight of formulation and the weight at sampling time, expressed as a percentage of the amount of IPA added to each. For the controls without IPA, percent weight loss was calculated relative to the amount of DEET in the original sample.

### Example 4—Structural Analysis

X-ray diffraction was used to study the interaction of DEET with the swelling clays. The d-spacing adapts to the intercalated molecule when adsorption occurs in the interlamellar spaces, giving rise to swelling of the clay structure. This is evidenced by changes in basal spacing of both the native and the HDTMA organo-clay before and after treatment with DEET as determined using X-ray diffraction. The degree of spacing change allows for possible deduction of the probable orientation of interlayer species.

X-ray diffraction patterns were recorded on a Siemens X-ray diffractometer D500 with a copper Kα radiator. The diffractograms were recorded from 2-30° 2θ in steps of 0.02 2θ, with a 1 second count time. The diffractometer was also fitted with a copper tube.

Basal spacing was used as a qualitative test for intercalation of HDTMA and DEET between the clay layers. Oriented DEET/clay residues of the samples studied by both GC and HPLC were prepared and analyzed, and compared to the matrix blanks. To study the effects of extraction on the DEET clay matrices, representative samples of the extracted residues were also analyzed before and after leaching.

The intensity data and peak location on the diffractograms showed first, second, and third order peaks. The intense primary peak was used as the reference peak for all comparisons. The difference in interlayer spacing between the blank clay sample and the sample treated with DEET was used to estimate the basal spacing due to adsorption of DEET.

### Example 5—Results for Evaporation Studies

IPA was evaporated from the samples to see if it had any effect on the incorporation of DEET onto the HDTMA treated clay. A student t-test was used to construct the confidence interval for the average amount of DEET incorporated into the HDTMA treated clays. Results in Table 1 show no consistent effect of IPA on sorption of DEET by both HDTMA treated clays. For 1×HDTMA clay with IPA, 29.3% DEET was incorporated, and in the absence of IPA 25% DEET was incorporated. For 3× HDTMA clay less DEET was incorporated in the presence of IPA (11.7%), compared to 16% in the absence of IPA.

TABLE 1
DEET retained in 1X and 3X HDTMA treated clays that had been
previously mixed with DEET, and then extracted with 6 ml of IPA.
Extent of intercalation expressed relative to amount of DEET added,
and normalized to the amount of clay used.

| | | | | % Inter- | |
|---|---|---|---|---|---|
| Sample | Volume | Average % | | calation | Basal |
| Exchanged | IPA | DEET | Standard | (mg DEET/ | Spacing |
| Clay ID* | (ml)^{#} | Intercalated | Deviation | mg clay) | (Å) |
| 1X | 2 | 29.3 | 9.5 | 58.6 | 29 |
| 1X | 0 | 25 | 12.8 | 50 | 30 |
| 3X | 2 | 11.7 | 13.8 | 23.4 | 28 |
| 3X | 0 | 16 | 9.2 | 32 | 29 |

*Each Sample contained 1 g of DEET and 0 mg HDTMA modified clay and varying amounts of IPA.
^{#}The added IPA was subsequently evaporated under nitrogen gas prior to re-extraction.

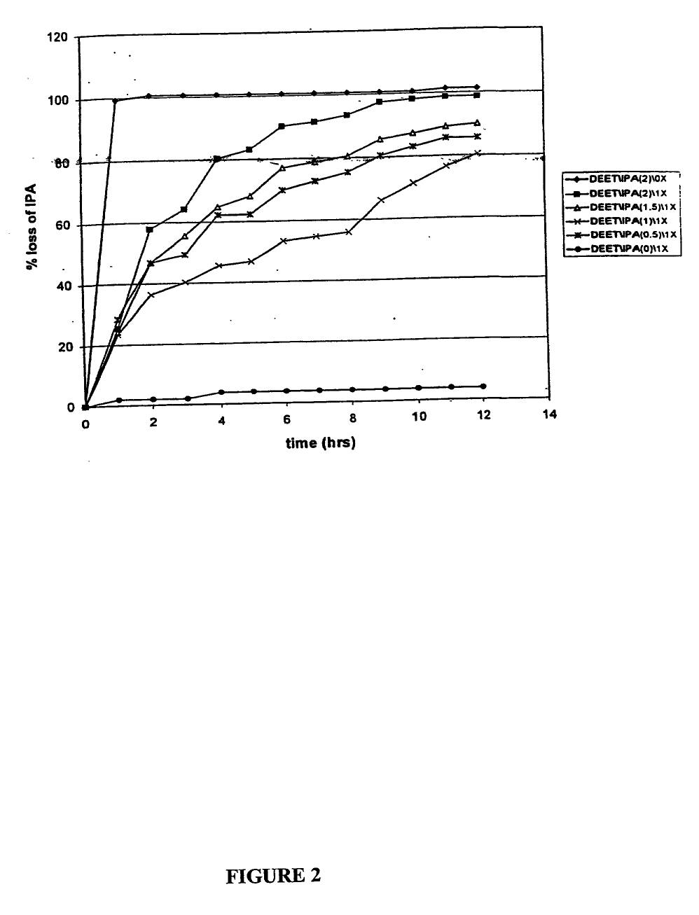

Several studies in literature allude to the fact that evaporation may be among the various routes responsible for the loss of DEET from the skin (Spencer et al., “Evaporation of Diethyltoluamide from Human Skin Invivo and Invitro,” Spencer et al., “Evaporation of Diethyltoluamide from Human Skin Invivo and Invitro,” Journal of Investigative Dermatology, 72:317-319 (1979); Robbins et al., “Review of the Biodistribution and Toxicology of the Insect Repellant N,N-Diethyl-m-toulamide (DEET)”, Journal of Toxicology and Environmental Health, 18:503-525 (1986), which are hereby incorporated by reference in their entirety). To further characterize this carrier system, evaporation studies were conducted to compare this property between the DEET in clay formulation, and pure DEET. Comparative weight loss before and after the samples were treated with nitrogen was used to develop evaporation kinetics of the matrix. FIG. 2 is a plot of percentage IPA loss versus time. For samples of technical DEET with no added IPA, there was only a 4% weight loss over 12 hours, suggesting that in the rest of the samples, the major weight loss ranging from 82-101% is likely due to evaporation of IPA from the samples.

### Example 6—Results for Structural Analysis

Table 2 displays the patterns of change in basal spacing in the native clay before and following treatment with either HDTMA alone or with DEET. Basal spacing for the 1×HDTMA clay and for the 3×HDMTA clay was 17Å and 20 Å, respectively. In the native clay, the change in basal spacing due to DEET was only 3 Å, compared to 10 Å as seen with DEET intercalated in both 1× and 3×HDMTA treated clays. Basal spacing due to 100 mg DEET was similar to that in samples treated with 1 g of DEET followed by up to 2 extractions with 6 ml IPA. Basal spacing therefore did not appear to be related to the amount of DEET, but rather to the orientation of DEET molecules within the interlayers, which is most likely similar in 1× and 3× clay.

TABLE 2
Effects of the various treatments on the basal spacing of the native clay.
Clay residues were analyzed by X-ray diffraction

| | BASAL SPACING (Å) | | |
|---|---|---|---|
| SAMPLE | Native Clay | 1X HDTMA Clay | 3X HDTMA Clay |
| clay* + water | 15 | 18 | 19 |
| clay* + IPA | | 18 | 20 |
| clay* + 30% IPA | 18 | 17 | 20 |
| Clay* + 30% IPA + | 21 | 28 | 32 |
| 0.1 g DEET | | | |
| clay^{#} + 1 g DEET | | 30 | 29 |
| only | | | |
| clay^{#} + 1 g DEET + | | 29 | 29 |
| 0.5 ml IPA | | | |
| clay^{#} + 1 g DEET + | | 29 | 29 |
| 1.0 ml IPA | | | |
| clay^{#} + 1 g DEET + | | 28 | 28 |
| 1.5 ml IPA | | | |
| clay^{#} + 1 g DEET + | | 29 | 28 |
| 2.0 ml IPAⁱ | | | |
| First extraction of | | 30 | 32 |
| sample 9ⁱⁱ | | | |
| Second extraction of | | 31 | 30 |
| sample 9ⁱⁱⁱ | | | |

ⁱSample 9, subsequently extracted.
ⁱⁱFirst extraction with 3 ml IPA and decanted.
ⁱⁱⁱSecond extraction with 3 ml IPA.
*All samples contained 250 mg of clay.
^{#}All samples contained 500 mg of clay.

### Example 7—Discussion Based on Examples 1-6

According to the results obtained, there was no consistent effect of IPA on the incorporation of DEET onto the clays. Even though the results in Table 1, showed a high degree of variation among samples, it was apparent that less than 30% of the added DEET was actually adsorbed by the clay under the preliminary experimental condition. Notably less DEET was incorporated into the 3× clay than the 1×. The amounts of DEET incorporated, normalized to the amount of clay used were 50% and 32% for the 1× and 3× clay, respectively. There is conflicting evidence in the literature with regard to the role of evaporation in the loss of DEET (Spencer et al., “Evaporation of Diethyltoluamide from Human Skin In vivo and In vitro,” Journal of Investigative Dermatology, 72:317-319 (1979); Robbins et al., “Review of the Biodistribution and Toxicology of the Insect Repellent N,N-Diethyl-m-toulamide (DEET)”, Journal of Toxicology and Environmental Health, 18:503-525 (1986), which are hereby incorporated by reference in their entirety) from the skin. The above data suggests that DEET does not evaporate readily (FIG. 2). However it is worth noting that there is a vast difference between laboratory and environmental conditions that conventional administration may be subject to, including the fact that the temperature of the skin is 37° C. as compared to 21° C. for room temperature.

Among the objectives of the above examples was to select an effective and DEET compatible extraction solvent. Based on the comparison between the correlation coefficients for the standard curves of DEET in IPA and in toluene, IPA was selected as the extraction solvent of choice for the analysis. IPA is also the solvent in which most commercial preparations of DEET are formulated.

Data from the preliminary experiments showed a high degree of variation within groups as far as the amount of DEET measured in the supernatant, even in aliquots from the same samples analyzed on the same day (Table 1). This would suggest additional sources of error elsewhere in the experimental design. These samples were either in suspension or semisolid form, which would make it very difficult to obtain homogenous samples. In addition, the amount of DEET used could have been too high as suggested by the low percentage of incorporation (Table 1), such that oversaturation of the clay would leave more on the surface, further compounding sampling by aliquots, and ultimately quantitation problems. For the characterization studies, which follow, universal spiking solutions were used to prepare multiple replicates of the DEET matrix that were extracted entirely. This was done to eliminate sampling by aliquots of potentially non-homogenous samples.

The recorded basal spacings for the 1× and 3×HDMTA treated clay were consistent with a model previously published. At an HDTMA load of 1 times the clay's CEC, the interlayer surfactant molecules formed bilayers and therefore gave a basal spacing of 18 Å. When the surfactant load was increased, the molecules were adsorbed as pseudotrilayers giving a higher basal spacing as seen with the 3×HDMTA clay. The structural analysis showed that both 1× and 3×HDMTA loaded clays treated with DEET exhibit the similar basal spacing due to DEET. This confirmed that in both 1× and 3× HDTMA loaded clay, DEET is interspersed between the layers, causing interlayer swelling. The results further suggested that the DEET molecules assume similar interlayer orientation in both cases. Comparison of interlayer spacing due to intercalation of DEET in native BP clay or HDTMA treated BP clay showed a d-spacing of 3 Å and 10 Å, respectively, however the change was not dependent on concentration of DEET used in the two DEET loadings reported here. The reason for the small interlayer expansion seen with native clay may not only be due to the fact that DEET is not adsorbed by this clay, but possibly the molecules that are adsorbed may assume an orientation different from those adsorbed in the HDTMA treated form. The basal spacing represents the swelling due to the molecule being intercalated in a certain orientation between the layers (White et al., “Pharmaceutical Aspects of Clay-Organic Interactions,” Md Eng. Prod. Res. Dev., 22:665-671 (1983), which is hereby incorporated by reference in its entirety), therefore the orientation of DEET molecules seemed to be independent of its concentration. Through X-ray analysis it was confirmed that one of the binding mechanisms involved interaction of DEET with the aliphatic HDTMA tails (Guangyao et al., “Co-sorption of Organic Contaminants from Water by Hexadecyltrirnrthylannonium Exchanged Clays,” Wat. Res., 30:1483-1489 (1996); Guangyao et al., “Enhanced Sorption of Organic Contaminants by Smectite Soils Modified with Catatonic Surfactant,” Journal of Environmental Quality, 27:806-814 (1998); Li et al., “Counterion Effects on the Sorption of Cationic Surfactant and Chromate on Natural Clinoptilolite,” Environmental Science and Technology, 31:2407-2412 (1997), which are hereby incorporated by reference in their entirety) which are also interspersed between the clay layers. The basal spacing did not change following the extraction with IPA of 60% of the added dose of DEET. There could be a several explanations for this observation. One is that extraction with IPA initially moves DEET molecules on the clay surfaces, which actually are not characterized by the X-ray method, and does not affect the DEET between the layers. Since basal spacing is a function of the orientation of interspersed species, it is appears that the orientation of intercalated DEET molecules does not change with DEET concentration.

### Example 8—Incorporation of DEET onto HDTMA Modified Clays

Following the results obtained in Examples 1-7, a new method for incorporating DEET onto the clay was developed.

The objective of this study was to find means of enhancing the affinity of clay for DEET and thereby increasing the intercalation of the chemical into the clay. In line with this objective, the influence of various HDTMA clay loading on the adsorptive properties of the clay for DEET was studied. Based on the hypothesis that HDTMA modified clays behave like a partitioning medium, the influence of the solvent on the extent of intercalation of DEET onto clay and the effects of increasing hydrophilicity of the solvent on the extent of intercalation was studied. An additional factor of aging the clay DEET matrix on the extent of intercalation was also studied.

The second objective was to characterize the selected matrix with regards to release kinetics and desorption under various solvent conditions.

In line with studying the effects of hydrophilicity of the solvent on the extent of intercalation of DEET onto the clay, a set of experiments was conducted to study the solubility of DEET in solvents containing water and IPA at varying proportions. These were later used as spiking solutions for incorporation of DEET onto the native and HDTMA treated clay.

Homogenous solutions of different concentrations of DEET with varying percentages of IPA in water were prepared and subsequently incorporated into the clay. Solutions of 30%, 40%, and 50% IPA in water were prepared. DEET was completely miscible with IPA, which was also completely miscible with water. The addition of DEET to the solvent mixture was therefore expected to produce a single homogenous phase within the limits of its solubility. Solutions were prepared consisting of the solvent systems and varying concentrations of DEET, from 12.5-200 mg/ml. DEET was added to the solvent in increments of 50 mg until the solution appeared turbid after vortexing for 5 seconds. Turbidity was used as a marker for saturation. The limit of DEET solubility for 30% IPA was 115 mg/ml, while for 40% and 50% IPA, the solubility limit was 200 mg/ml.

HDTMA bromide was dissolved in warm distilled water and intercalated with the native clay at 0.5× the clay's CEC, to prepare the organo-clay. Native clay was hydrated by mixing 10 g in 80 ml of water with a resultant gel that was allowed to stand overnight at room temperature. The native clay gel was then treated by adding 80 ml of the HDTMA bromide solution (0.047M), resulting in flocculation. The clay mixture was placed in the oven overnight at 60° C., followed by centrifugation at 3000 rpm for 10 minutes. The supernatant was discarded and the HDTMA clay was washed with 100 ml distilled water two times and was washed a third time by placing the sample in the oven at 60° C. with the third wash water. The supernatant was discarded and the wet clay was quick-frozen and then freeze-dried, and stored for later use at room temperature. The freeze-dried 0.5×HDMTA clay appeared to be granular and clumped. Part of the HDTMA organo-clay was gently ground with mortar and pestle and is hereafter referred to as 0.5× Fine. The remaining clay was used as prepared.

Both 1× and 3× clays were supplied by the Department of Geology at SUNY Buffalo. The procedure used was similar to that for loading 0.5×HDMTA clay, except that the native BP clay was not hydrated prior to adding the HDTMA solution. A detailed procedure for loading these clays with HDTMA is outlined in U.S. Pat. No. 6,015,816, which is hereby incorporated by reference in its entirety.

Untreated and HDTMA treated clays 0.5×, 1×, and 3× (250 mg) were weighed into 7 ml glass vials with Teflon lined caps. Two milliliters of the DEET spiking solutions were prepared as described above. The mixtures were vortexed for 30 seconds, until suspended. The samples were then allowed to mix on an end over end rotary mixer at 50 rpm, for 24 hours at room temperature. The samples were allowed to stand for 8 days after which they were centrifuged at 1800 rpm for 15 minutes. The supernatant was analyzed using HPLC to determine the equilibrium DEET concentration, and thereby obtain the amount retained in the clay as a result. The residue was later analyzed by X-ray diffraction in order to study the interlayer interaction of DEET with clay.

### Example 9—Release of DEET Incorporated Into the Organo-clays

Sorption of a DEET by clay may leads to a number of desirable effects, among which are controlled release of DEET and prevention of DEET absorption topically. Data from desorption studies is therefore important to the understanding of the behavior of the system at different times following the administration, and the release relative to the volume of the body fluids in the surrounding environment.

A study of the fraction of DEET released from its matrix system as a function of time after introduction of a solvent was performed in order to quantitate DEET's release profile. This is a critical physical characteristic of any drug immobilizing system. The formulations studied for leachability were prepared using DEET in 30% IPA and 1×HDTMA since this was proved the combination which exhibited the highest DEET sorption capacity. Two systems were studied: a dialysis membrane loaded with the DEET equilibrated clay, and the direct application of solvent to the same matrix.

Leachability Through Dialysis Membrane

After reviewing various options for studying this characteristic, the membrane diffusion technique was chosen. The primary advantage of dialysis is that the membrane physically separates the solid phase from the solvent eliminating issues related to fine retention by the solvent. The matrix is separated from the bathing solution by a dialysis membrane, which is selectively permeable to low molecular weight compounds such as DEET, and allows DEET to diffuse out of the matrix suspension, across the membrane, and into the bathing solution. The other advantages of this system are that it is cleaner, it does not require centrifugation for separation, and therefore would allow for reduced sampling intervals if required.

Samples of DEET in 30% IPA at concentrations of 12.5 and 25 mg/ml were prepared with either 250 mg of the respective clay or with no clay in accordance with the incorporation procedure described above. Dialysis membranes were also prepared as described in Example 1 above. The DEET/clay suspension was qualitatively transferred into the dialysis tubing. The dialysis tubing was tied and suspended in a bathing solution of 30 ml of 30% IPA in 40 ml glass bottles with tops. The bottles were allowed to rotate on the rotary mixer for the entire duration of the experiment. Ten microliter aliquots of the bathing solution were collected at 0 min., 15 min., 30 min., 1 hour, 2 hours, 4 hours, 6 hours, 18 hours, and 25 hours and subjected to HPLC analysis for DEET concentration.

DEET Removal from Clays Directly into Solvent

Due to constraints experienced with studying the carrier's time release profile using the dialysis technique, a system where the clay was incubated directly into the solvent was used. This was also used to further characterize the extent of release of DEET from the system relative to various solvent types and varying volumes of solvents.

To evaluate leachability under extreme conditions, i.e, in a solvent in which DEET is highly soluble, samples were treated with 30 ml of 30% IPA. Samples were allowed to mix on a rotary mixer for time periods of 0 min., 30 min., 1 hour, 2 hours, 3 hours, 4 hours, 6 hours, and 8 hours. Samples were centrifuged and aliquots of the supernatant were collected and analyzed for DEET by HPLC.

To study removal of DEET from the clay matrix under conditions which simulate the skin surface, 0.9% sodium chloride solution was used in place of the solvent to simulate sweat to leach DEET from the matrix. Samples were extracted with 30 ml of normal saline, following the same experimental procedure as above; and for the same time points.

The release of a DEET from the carrier system may be influenced by a number of different processes among which is the immediate surrounding to which it is placed. Volume could be an important determinant of DEET release from the matrix since if it were to be administered on the skin, the volume of sweat it comes into contact with is likely to influence its release characteristic.

IPA is a solvent in which DEET is highly soluble. Since solubility would be an important factor in deciding the vehicle in which a topical formulation should be dispersed, studies were performed to assess the role of increasing volumes of IPA on DEET removal from the clay matrix. Spiking solutions of DEET were prepared in 30% IPA to give a final concentration of 12.5 mg/ml and 50 mg/ml. Two milliliters of the spiking solutions were incorporated into 250 mg of 1×HDTMA treated clay and kept at room temperature until time of extraction.

For leachability, 10 ml of 30% IPA was added, mixed in with the suspension for 45 minutes on the rotary mixer, centrifuged, and supernatant collected for analysis by HPLC.

The 30% IPA was added in increments of 10 ml to a final volume of 30 ml. DEET removal from the clay was assessed at each 10 ml increment.

Sodium chloride was used as a surrogate for sweat. Samples were prepared according to the procedure as described above. An identical extraction paradigm was used and DEET release was determined after each successive addition of 0.9% sodium chloride.

### Example 10—Effects of Successive Washings on DEET Release from the Carrier

To analyze the ability of the carrier to retain its drug load following repeated washes, the supernatant for all samples from the previous studies was removed by decanting the 30 ml of IPA or normal saline, and instantaneously replacing it with 10 ml of the respective solvents for two successive washes. Ten microliters of the supernatant was collected from each wash and analyzed by HPLC. The results (Table 2) indicated that saline was inefficient for leaching out DEET from the matrix as compared to IPA. To determine whether this was a solubility factor or some effect that DEET had on DEET binding to clay, all the samples were further washed in 30% IPA for two successive washes of 10 ml each.

### Example 11—Aging

Chemical incubation with soils and clays has been shown to exhibit an aging phenomena (Turkall et al., “The Effects of Aging in Soil on Dermal Bioavailability of Mercury,” The Toxicologist 38^{th} Annual Meeting, 48 (1999), which is hereby incorporated by reference in its entirety). This influences the bioavailability of the chemical when exposure due to bound chemicals occurs.

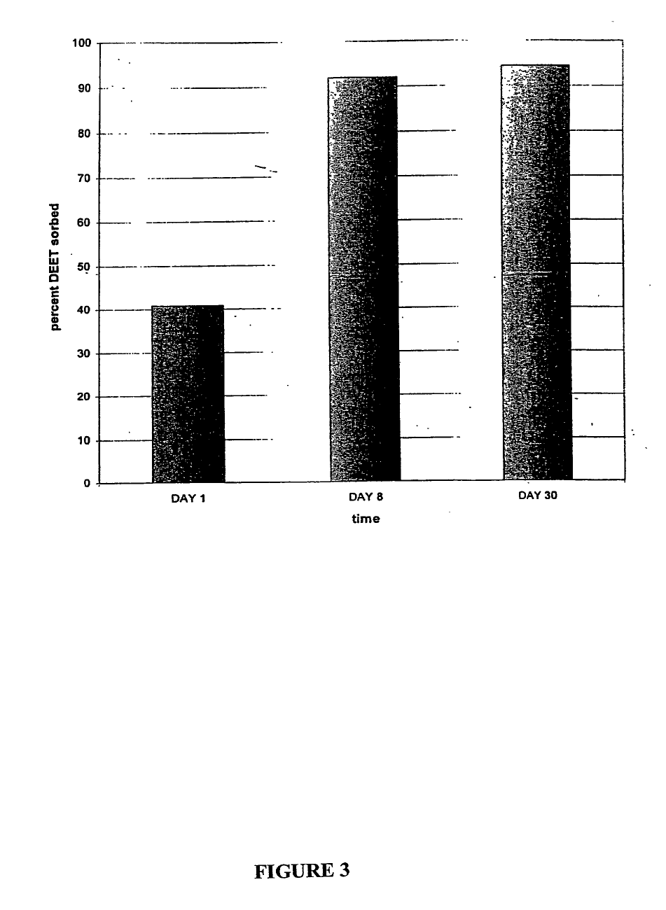

To study what effect aging may have on adsorption and binding of DEET to the clay, samples were allowed to stand in closed containers at room temperature (20-23° C.) for various time periods. Samples were then extracted as described above. Equilibrium concentrations of DEET in supernatant were studied at 24 hours, 8 days, and 30 days after incorporation (see FIG. 3).

### Example 12—Analysis of Samples

Once various solvents were mixed with DEET, turbidity of the samples was used as a marker for saturation. All DEET spiking solutions were below saturation point.

An HPLC method established for Examples 1-7 was also used for quantitation of DEET in this study.

To measure the extent of incorporation, samples were centrifuged, and the supernatant was analyzed by HPLC to estimate the amount of DEET sorbed at equilibrium. For those samples that were dialyzed, 10 μl of the solution was collected without centrifugation.

The amount of DEET sorbed on the various clays was calculated.

Total recovery of DEET from the matrix following repeated washes was calculated by adding amounts appearing in each wash, subtracting this cumulative amount from the initial amount added, resulting in the amount retained by the clay. All sorption experiments were performed in duplicate and the means were plotted.

An X-ray diffraction method established in Example 4 was also used for qualitative analysis of 0.5×, 1×, and 3×HDMTA clay residues before and after treatment with DEET.

Residues from 0.5×, 1×, and 3×clay were analyzed by X-ray diffraction before and after treatment of the clays with DEET. For the 0.5× clay, the clay particles were either ground (fine) on the mortar and pestle, or used as is for incorporating DEET. Both sets of DEET/0.5×HDMTA/IPA samples were to be analyzed using X-ray diffraction. The samples had been allowed to stand at room temperature for three months, and the clay residues for both sets of 0.5× clay treated with DEET separated. They formed two distinct clay layers, the top one being lighter in color and also consisting of finer particles than the bottom layer. The two layers were X-rayed separately for each set of samples.

The same qualitative analysis procedures used in Example 4 for characterization of the interaction of DEET molecules with HDTMA treated clay were adopted.

### Example 13—Visual Analysis of Solubility of DEET in IPA and Water

Varying proportions of IPA and water were evaluated with regard to their ability to dissolve DEET. 30% IPA in water could only dissolve up to 115 mg/ml of DEET. For the 40% and 50% IPA in water, concentrations of as much as 200 mg/ml could be dissolved. All three solvents were further evaluated for their effects on the adsorption of DEET onto the various clays.

### Example 14—Quantitation of DEET with HPLC

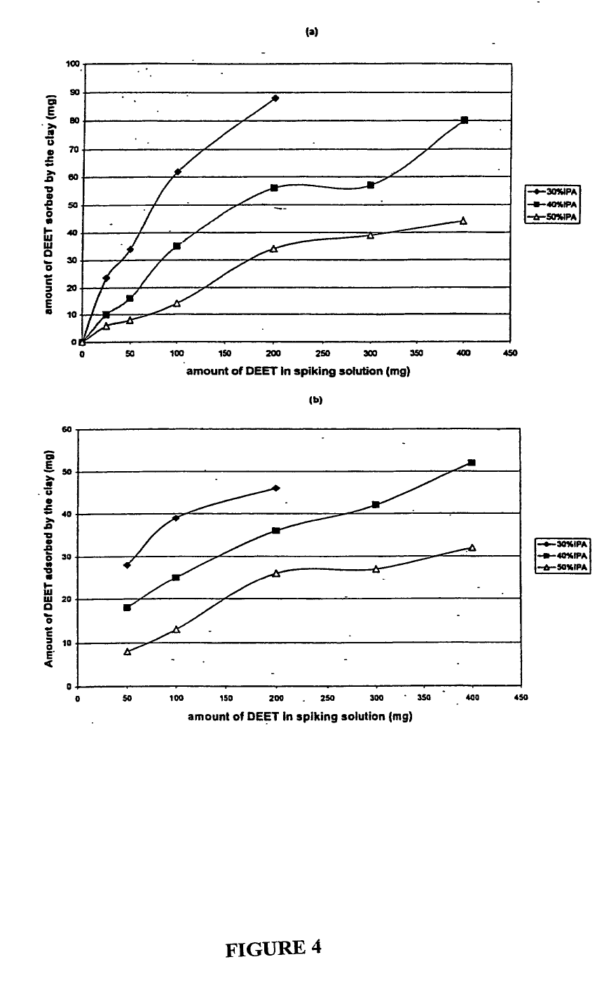

FIGS. 4A-B shows how the solvents for the spiking solutions affected the extent of intercalation of DEET onto the 1×HDMTA clay. It is clear that the type of solvent used significantly influenced the extent to which DEET would be adsorbed from solution to the solid clay matrix. The same trend was seen with the 3×HDMTA clay. The presence of water enhanced adsorption as shown by the increase in sorption in the range of 50-55% based on the uptake of the same sample in 30% IPA compared to 50% IPA.

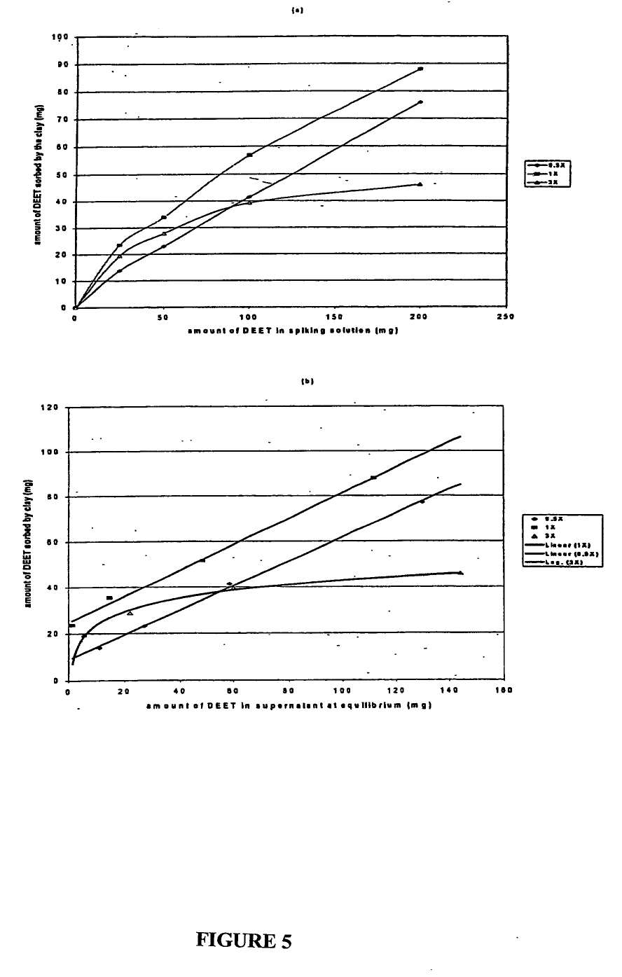

The sorptive isotherms for DEET onto clays exchanged with HDTMA at 0.5, 1, and 3 times the CEC (0.5×, 1×, and 3× respectively) are presented in FIGS. 5A-B. The results show that all the clays are able to adsorb DEET. The clays differed in their capacities for DEET adsorption. Sorption isotherms for 1× and 0.5×HDMTA treated clays showed similar overall curves. In contrast, adsorption of DEET by 3×HDMTA treated clay resulted in an isotherm with distinct curvature that differed in shape and extent from 0.5× and 1×. The isotherm for both 1× and 3× showed a much sharper rise in uptake at concentrations below 12.5 mg/ml of DEET in solution. However at higher concentrations of DEET, the isotherm for 3× leveled off in a manner that suggested saturation of sorptive mechanisms. The isotherm for 0.5× at concentrations above 12.5 mg/ml of DEET paralleled that of 1× over the same concentration range. Isotherms for 0.5× and 3× exhibited a crossover at concentrations above 25 mg/ml. Above a concentration of 12.5 mg/ml, the isotherms of 0.5× and 1× paralleled each other, with the slopes at 1.15 and 1.16, respectively. The slopes were used to estimate the adsorption coefficient of these clays for DEET, and since they were linear, the value was constant over the concentrations studied. Overall, 1×HDMTA treated clay showed superior sorption characteristics for DEET, followed by 0.5×, while 3× exhibited the weakest sorption characteristics of the three. The sorption profile for 3× appeared to plateau at high DEET concentrations.

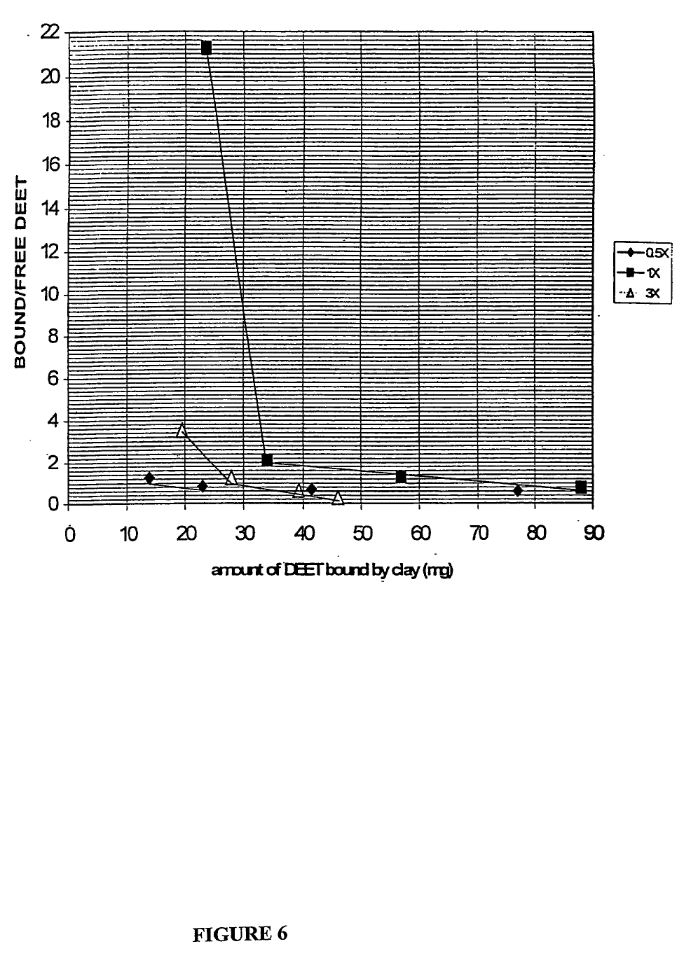

The Scatchard plot has been used extensively in the past to analyze the binding of chemicals to plasma proteins. From this analysis the number of binding sites per molecule of protein and the affinity constants of protein ligand complex can be determined. In addition, it frequently exhibits non-linearity indicating the presence of more than one binding mechanism. The Scatchard plot was adopted to further support the theory that there is more than one mechanism responsible for the binding of DEET to the clay. FIG. 6 is a Scatchard plot of the sorption data. The plots also exhibit non linearity, and the slopes are different for the three clays in line with the fact that all three of them have different affinities and capacities for binding DEET. To further support the theory that for 3×, one of the mechanisms may be saturable at high concentrations of DEET, the following observations were made. At lower concentrations, 1× clay has the highest ratio of bound-to-free DEET of 21.25, followed by 3× at 3.52, and 0.5× at 1.25. On the other hand, at high concentrations of DEET in the sample, 1× still has the highest ratio of 0.78, followed by 0.5× at 0.61, and lastly 3× at 0.29.

Based on the results above, using 30% IPA as solvent for the liquid phase and I×HDTMA treated clay as solid adsorptive phase seemed to provide the highest degree of intercalation for DEET, as compared to any other combination. It was in this light that this particular matrix was chosen to characterize further, and to evaluate its desorptive properties.

To study the release profile of the DEET carrier system, the fraction of DEET released as a function of time was evaluated, once the system was given an unrestricted opportunity to release its DEET load to the surroundings.

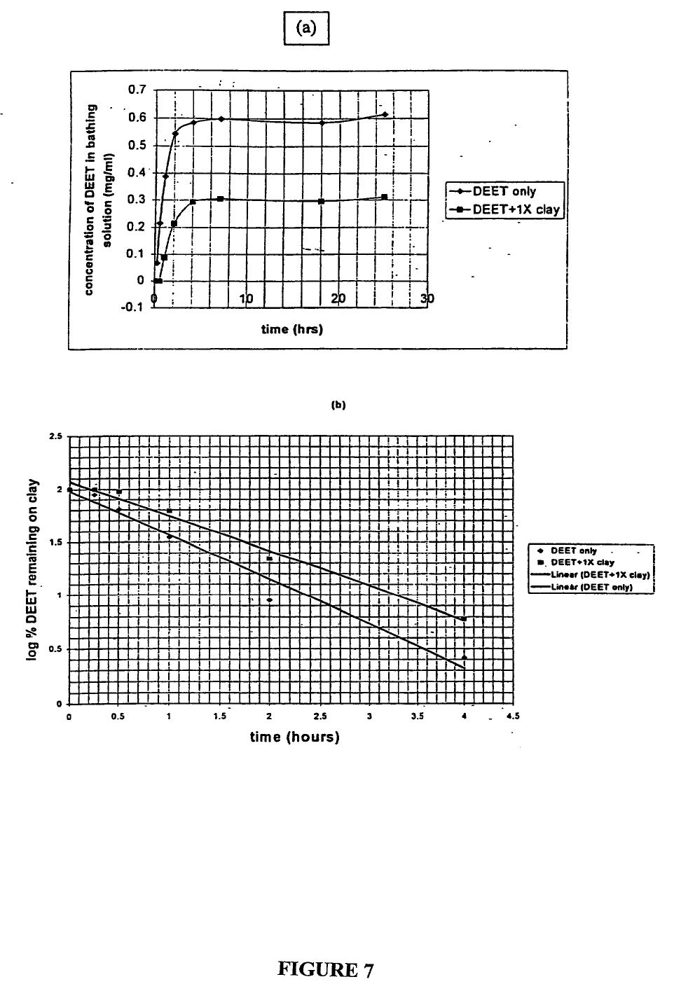

Equilibrium dialysis was performed in which the DEET/clay matrix was separated from the reservoir of extraction solvent by a dialysis membrane. The results are shown in FIGS. 7A-B. Although a quantitative transfer of material into the dialysis membrane was attempted, not all the suspension could be quantitatively transferred to the membrane, thus firm conclusions could not be drawn concerning the exact fraction of DEET extracted. FIG. 7A is an arithmetic plot of the concentration of DEET in the bathing solution relative to time. This data compares the time release pattern between the free solution of DEET and that in the clay matrix. FIG. 7B is a logarithmic plot of the percent of DEET remaining on the clay relative to time. This was used to calculate the half-life for the release of DEET from the matrix. Half-life is the time required for the concentration of DEET in the HDTMA/clay matrix to decrease by half. Percentage of DEET remaining was calculated and plotted for comparative purposes. Based on the slope of the curves in FIG. 7B, the half-lives for free DEET in IPA and DEET in the HDTMA/clay matrix were 43.8 and 53.3 minutes, respectively. The formulation of free DEET in IPA was expected to equilibrate relatively instantaneously with the bathing solution. The exaggerated half-life seen here is most likely the due to barrier effect; DEET had to transverse the dialysis membrane before equilibrating with the bathing solution. The 43.8 minutes half-life for the free DEET in IPA was used as the barrier factor and was deducted from the half-life for DEET in HDTMA/clay matrix observed under similar conditions. The actual half-life for DEET in HDTMA/clay matrix was therefore 53.3 minus 43.8 minutes, or 9.5 minutes.

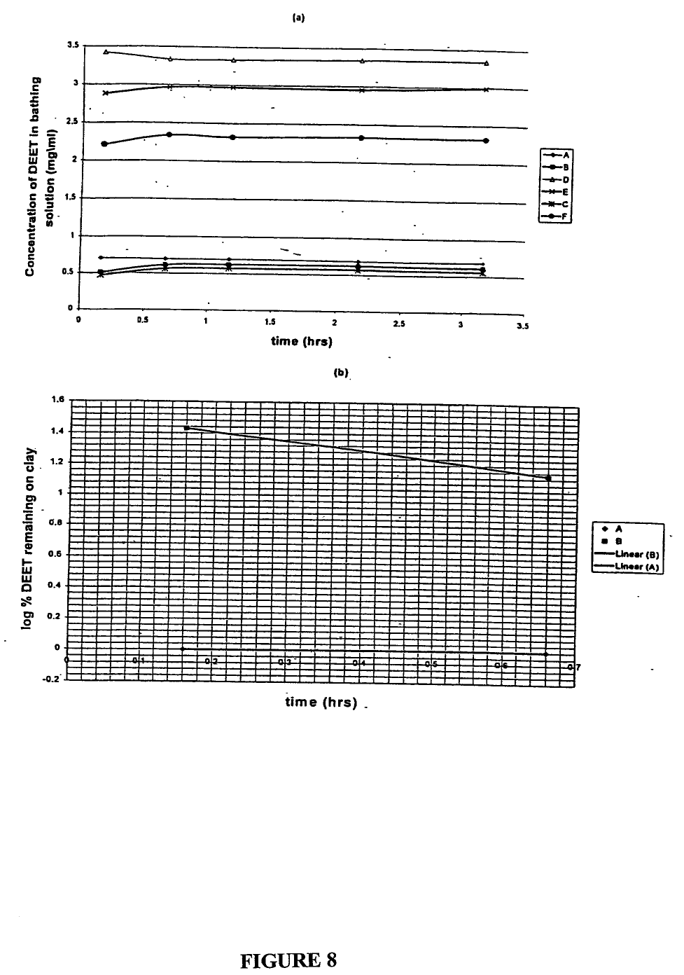

To directly assess the half-life and compare it to the equilibrium dialysis method above, DEET treated 1× clay was equilibrated with 30 ml of solvent in the absence of dialysis membrane and sampled at intervals (FIGS. 8A-B). DEET released from the HDTMA/clay equilibrated with the solvent within 40 minutes of IPA addition. As expected, free DEET without the clay matrix equilibrated instantaneously with the solvent. FIG. 8B displays a plot of the percentage of DEET remaining on the clay after dilution into 30 ml at various time intervals. Only the formulations containing 25 mg of DEET were used to estimate the half-life of DEET in the formulation. At this concentration, over 92% (FIG. 5A) of DEET was associated with the HDTMA clay. From the slopes, the half life of DEET in the formulation was 31 minutes, which was not in agreement with the estimates arrived from the dialysis study. The carrier system exhibited similar time release profiles when extracted into of normal saline. In the absence of the dialysis membrane, the sampling interval had to be increased to allow for centrifugation. In addition, the earliest time point was at 10 minutes. This may explain the exaggerated half-life observed.

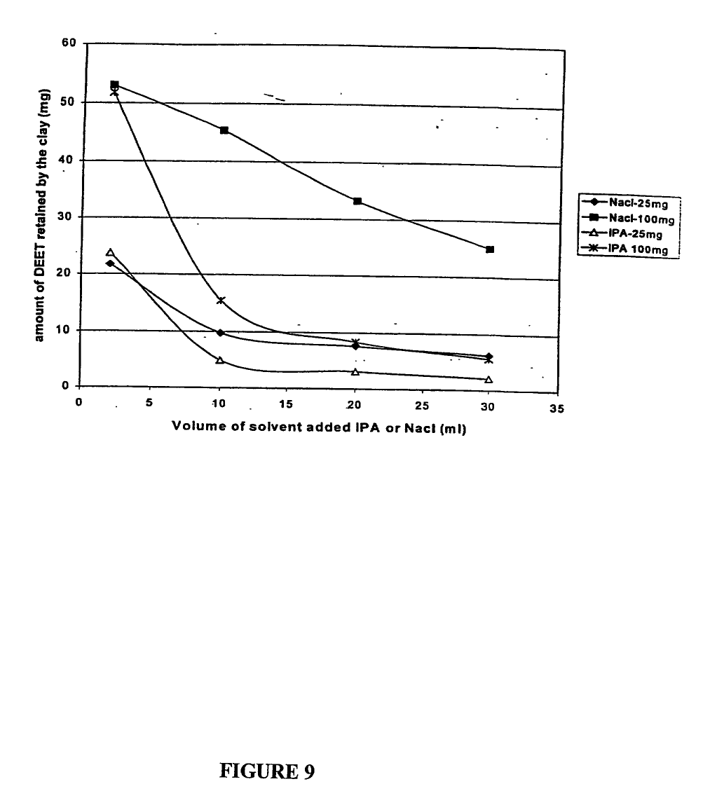

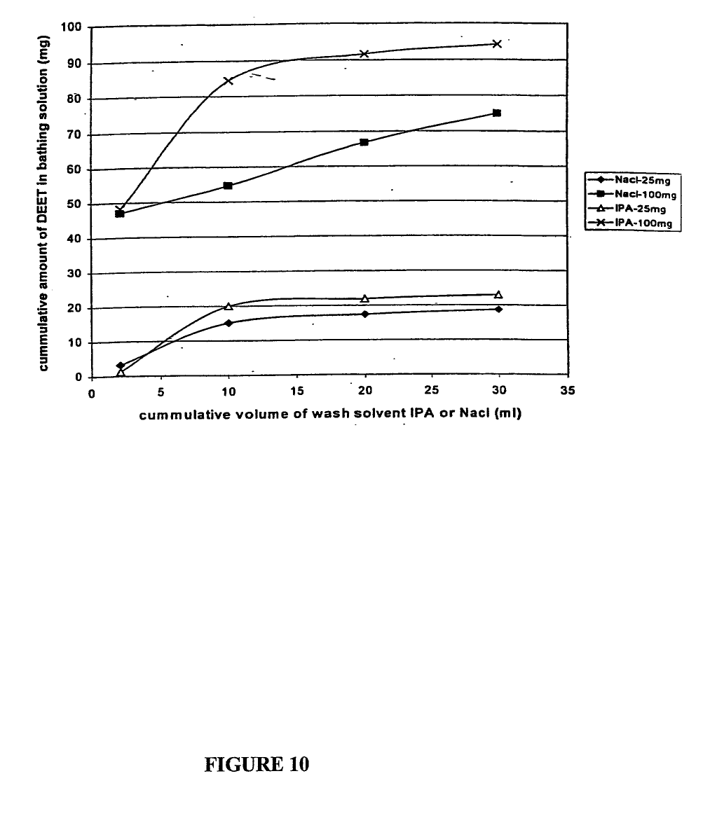

The effect of solvent volume on desorbability of DEET from HDTMA treated clays was evaluated by consecutively diluting the matrix formulation having different DEET loading levels with either 30% IPA or normal saline. Increasing volumes of the wash solvent were used without decanting prior to subsequent dilutions. In FIG. 9, a plot of the amount of DEET retained against the volume of either wash solution, shows that the more DEET incorporated in the clay, the higher the volume of solvent it takes to leach out the DEET. For all the IPA washes, the first 10 ml caused a large release of DEET from the matrix into the solution followed by a more gradual release. On the other hand, the saline wash showed a slower initial release which is retained all throughout the volumes studied. When normal saline was used, at least 50% of the initial amount was retained at 30 ml of rinse volume, compared to 16% for IPA. This suggested that both the nature of the wash solvent and volume were important determinants in desorption of DEET from the carrier. FIG. 10 expresses the same data as cumulative percent loss of DEET versus volume of wash solvent.

The total recovery of DEET into the solvent was evaluated by washing the matrix and decanting the supernatant, and replacing the volume until DEET could no longer be detected in the supernatant. Table 3 shows the results of the successive washes. As expected, it took considerable washing to extract DEET at higher concentrations than from the lower ones. The fact that for some of the samples approximately 97% recovery was documented discounts losses due to instability of DEET, in either the solvent or the matrix. Normal saline does not cause as much leaching of DEET from the matrix, compared to 30% IPA. Once it became apparent that normal saline had a reduced ability to extract DEET from matrix, those samples that had been previously washed in saline solution were then washed further in IPA 30%. The results show that IPA did leach out the residual DEET to a greater extent and at a faster rate (Table 3).

TABLE 3
Comparative leachability of DEET from 1X HDTMA clay, following
extraction with either 30% IPA or 0.9% NaCl. Wash and decant.

| Amount of DEET added | | | | |
|---|---|---|---|---|
| to sample | 25 mg | 25 mg | 100 mg | 100 mg |
| Extraction Solvent | IPA | NaCl* | IPA* | NaCl* |
| WASHING | % DEET RETAINED BY CLAY | | | |
| CONDITIONS | (mg DEET/mg clay) | | | |
| Add 30 ml wash solvent | 0.84 | 2.5 | 2.28 | 9.64 |
| Decant and add 10 ml | 0.84 | 2.46 | 1.32 | 6 |
| Decant and add 10 ml | 0.84 | 2.46 | 1.12 | 4.08 |
| Decant and add 10 ml | 0.84 | 1.02 | 1.12 | 1.52 |
| IPA to all samples | | | | |
| Decant and add 10 ml | 0.84 | 1.02 | 1.12 | 1.24 |
| IPA to all samples | | | | |
| Basal Spacing (Å)* | 19.2 | 18.5 | 20.3 | 20.4 |

*Residues from the final washes were analyzed by X-ray diffraction

Upon physical examination of the samples during the wash process, the following observations were made. For those samples washed in normal saline, there appeared to be some dispersion when wash solvent was added to matrix. In addition, once they were allowed to mix for a few hours, a foamy layer appeared on top of the supernatant, and this was also accompanied by some clay particles that would not sediment even after centrifugation at 1800 rpm for 15 minutes. These clay particles actually formed a floating layer separate from the rest of the clay residue, suggesting that they no longer had the same density as the residue, probably due to the displacement of HDTMA.

### Example 15—Structural Analysis by X-ray Diffraction

Table 4 illustrates the results of the structural analysis of the clay residues by X-ray diffraction. Comparing the basal spacing of all three HDTMA treated clays used in the experiments, 3×HDMTA clay exhibited the highest degree of expansion due to the interlayer intercalation of HDTMA. The 0.5× clay resulted in the least expanded clay structure. Analysis of the different fractions of the 0.5× clay showed similar basal expansion due to DEET for both the bottom and the top components of the residue only in the set of samples where the clay had been refined by mortar and pestle. For the set where the 0.5× clay was used as is, the top layer of the residue seemed to have expanded more than the bottom layer. In general DEET seemed to cause the same extent of interlayer expansion in 0.5×, 1×, and 3×.

TABLE 4
Effects of DEET on the basal spacing of clays loaded with
HDTMA to 0.5X, 1X, and 3X its CEC.

| | BASAL SPACING (Å) | | | | | | |
|---|---|---|---|---|---|---|---|
| | 0.5X HDTMA clay | | | | | | |
| | Fine* | | Coarse⁺ | | 1X HDTMA clay | | 3X HDTMA |
| SAMPLE | B^{#} | T^{&} | B^{#} | T^{&} | IPA | NaCl | clay |
| Clay^{Ψ}/30% IPA | — | | — | 16 | 17 | 17 | 20 |
| Clay^{Ψ}/30% | 28 | 29 | 17 | 29 | 28 | 28 | 32 |
| IPA/0.1 g DEET | | | | | | | |
| Clay^{Ψ}/residual | — | — | — | — | 18.5 | 19.2 | — |
| DEET (0.025 g) [symbol] | | | | | | | |
| Clay^{Ψ}/residual | — | — | — | — | 20.3 | 20.4 | — |
| DEET (0.1 g) [symbol] | | | | | | | |

*The HDTMA clay was finely dispersed in the mortar and pestle prior to incorporating DEET.
⁺DEET was added to clay as is.
^{#}Analysis from the bottom component of the residue.
^{&}Analysis from the top component of the residue. [symbol]
All samples were washed in 30% IPA until no DEET could be detected in the supernatant, see Table 3 for details.
^{Ψ}samples contained 250 mg of the respective clay.

### Example 16—Discussion Based on Examples 8-15

Although there are reports in literature on the repellent effects and kinetics of DEET, no studies are available regarding its bioavailability following adsorption to native or treated clays. The data from this study demonstrates that the presence of clay produces differences in the availability of DEET after incorporation into the carrier.

HDTMA sorbed onto the clay acts as a bulk partitioning organophilic phase. The movement of DEET from one phase to the other is dependent on its affinity for either phase. DEET is a lipophilic molecule with high solubility in IPA. DEET is expected to associate preferentially with the liquid IPA in which it is freely soluble as opposed to the solid clay phase. This could have also contributed to the low sorption percentages as seen in Table 1 for the studies described in Examples 1-7.

Addition of water to IPA renders the solution more hydrophilic. This results in a decreased affinity of DEET for the liquid phase. This would enhance the sorption of DEET by the organophilic clay. Using various water:IPA solvent combinations, solvent related differences were observed in the extent to which DEET was sorbed onto the clay. The highest amount of sorption by the HDTMA clay was seen with 30% IPA, while 50% IPA exhibited the lowest. This increase in adsorption can be explained by the likely lowering of the vehicle to clay partition coefficient. The degree of uptake of DEET by the organophilic clay was inversely proportional to its solubility in the liquid phase. This property is also useful for future consideration of the actual vehicle that the carrier will be formulated in.

The affinity of DEET for the organophilic clays was reflected by the reduced recovery of added DEET from clay samples that contained HDTMA clays. The amount of HDTMA in the clay affected the adsorption of DEET. As expected, the native clay was an ineffective sorbent for DEET under the experimental conditions used. DEET sorption by HDTMA modified clays displayed non-linear isotherms (FIGS. 5A-B). This suggests that multiple sorbate-sorbent interaction(s) in addition to partitioning are involved. The data from the Scatchard plots further support this hypothesis. These results were consistent with those observed in recently published studies (Guangyao et al., “Co-sorption of Organic Contaminants from Water by Hexadecyltrirnrthylannonium Echanged Clays,” Wat Res, 30:1483-1489 (1996); Guangyao et al., “Enhanced Sorption of Organic Contaminants by Smectite Soils Modified with Cationic Surfactant,” Journal of Environmental Quality, 27:806-814 (1998), which are hereby incorporated by reference in their entirety) for the sorption of aromatic compounds by HDTMA modified clays. Proposed mechanisms for the sorption of aromatic compounds by HDTMA modified clays include solute partitioning, solvation of the cation ammonium centers, the alkyl chains of the HDTMA, and the mineral surfaces (Boyd et al., “Layer Charge Characteristics of 2:1,” Clay Minerals, Vol. 6.; Guangyao et al., “Co-sorption of Organic Contaminants from Water by Hexadecyltrirnrthylannonium Echanged Clays,” Wat Res, 30:1483-1489 (1996); Guangyao et al., “Enhanced Sorption of Organic Contaminants by Smectite Soils Modified with Cationic Surfactant,” Journal of Environmental Quality, 27:806-814 (1998), which are hereby incorporated by reference in their entirety).

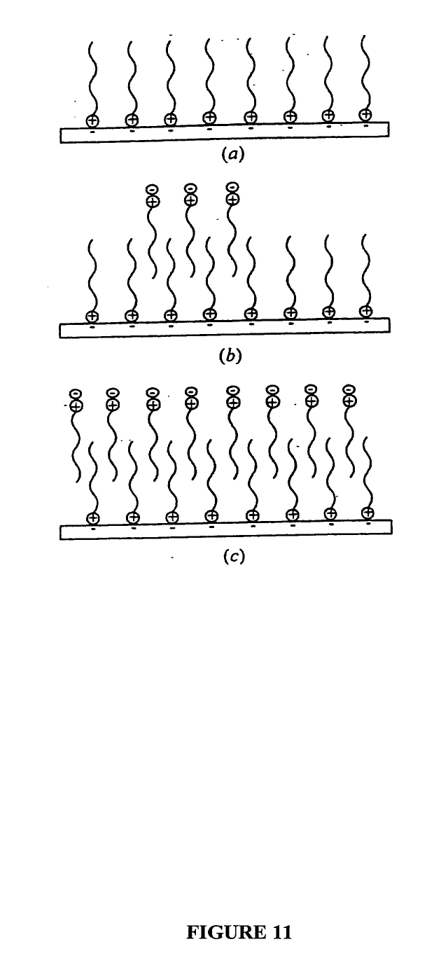

In characterizing the sorption of HDTMA onto negatively charged clays it was reported that sorption of HDTMA by clays involve both cation exchange and hydrophobic bonding (Li et al., “Sorption of Chromate and PCE by Surfactant-Modified Clay Minerals,” Environmental Engineering Science, 15:237-245 (1998); Guangyao et al., “Enhanced Sorption of Organic Contaminants by Smectite Soils Modified with Cationic Surfactant,” Journal of Environmental Quality, 27:806-814 (1998); Guangyao et al., “Mechanism(s) Controlling Sorption of Neutral Organic Contaminants by Surfactant Derived and Natural Organic Matter,” Environmental Science & Technology, 30:1553-1557 (1996), which are hereby incorporated by reference in their entirety). At lower HDTMA loading levels, adsorption of HDTMA molecules onto the clay surfaces forms monolayers with cation exchange as the major mechanism (see FIGS. 11A-C). At this lower surfactant loading level, below 0.7 CEC, very little HDTMA will be distributed on the external surfaces of the clay (Xu et al., “Alternative Model for Cationic Surfactant Absorption by Layer Silicates,” Environ. Sci. Technol., 29:3022-3028 (1995) which is hereby incorporated by reference in its entirety). At higher loading levels, above 0.7 CEC, both cation exchange and hydrophobic bonding occur concurrently resulting in the formation patchy or complete bilayers (Boyd et al., “Layer Charge Characteristics of 2:1,” Clay Minerals, Vol. 6; Li et al., “Sorption of Chromate and PCE by Surfactant-Modified Clay Minerals,” Environmental Engineering Science, 15:237-245 (1998); Guangyao et al., “Co-sorption of Organic Contaminants from Water by Hexadecyltrirnrthylannonium Exchanged Clays,” Wat Res, 30:1483-1489 (1996); Guangyao et al., “Enhanced Sorption of Organic Contaminants by Smectite Soils Modified with Cationic Surfactant,” Journal of Environmental Quality, 27:806-814 (1998); Guangyao et al., “Mechanism(s) Controlling Sorption of Neutral Organic Contaminants by Surfactant Derived and Natural Organic Matter,” Environmental Science & Technology, 30:1553-1557 (1996), which are hereby incorporated reference in their entirety). At even higher concentrations of HDTMA, the interlayer surfactant molecules may form pseudotrilayers. Results of the X-ray analysis for both the 1× and 3× were consistent with available literature in that they form bilayers and pseudotrilayers, respectively. The basal spacing for 0.5× clay was 16 Åand was higher than the spacing due to monolayer arrangement of the surfactant molecules. This seemed to suggest that even at this CEC, there was some formation of bilayers.

Results from this study demonstrate that at all concentrations studied, 1×HDTMA clay was superior to both 0.5× and 3× clays with regards to sorption of DEET (FIGS. 5A-B). This difference was likely related to the orientation and distribution of HDTMA molecules on the clay surfaces. Comparing the curvature of the isotherms for DEET incorporated onto the various clays (FIGS. 5A-B), they all showed initial slopes at lower concentrations that are steeper than those at higher concentrations of DEET. The steepness of the initial slopes ranged in the order of 1×, 3×, and 0.5×, with 1× being the steepest. At higher concentrations, all the slopes were less steep with respect to the initial ones. Both 1× and 0.5× had parallel slopes with gradients of 1.16 and 1.15 respectively. The slope for 3× at higher concentrations of DEET plateaued off, suggestive of saturation. The differences in the slopes at different concentrations of DEET could be attributed to the fact that different binding mechanisms for DEET onto clay occur concurrently at lower concentrations of DEET, while one is inhibited at higher concentrations. The tailing off observed with the isotherm for 3× at elevated concentrations of DEET further supports this theory. The parallel isotherms for 1× and 0.5× at higher concentrations of DEET suggested that for both clays similar binding mechanisms were operational to the same extent in this concentration range. The difference between the slope for 3× and 1× or 0.5× suggested that one of the mechanisms involved at lower concentrations became inhibited or saturated at higher concentrations. The sorption capacity of 3× clay for DEET may be limited at higher concentrations by stearic hindrance. At 3×HDMTA loading level, the clay became oversaturated with HDTMA molecules which caused a build-up of positive charge on the clay surfaces (Guangyao et al., “Co-Sorption of Organic Contaminants from Water by Hexadecyltrirnrthylannonium Exchanged Clays,” Wat Res, 30:1483-1489 (1966); Guangyao et al., “Enhanced Sorption of Organic Contaminants by Smectite Soils Modified with Cationic Surfactant,” Journal of Environmental Quality, 27: 806-814 (1998); Rakhshandehroo et al., “Hydraulic Characteristics of Organo Modified Soils for us in Sorptive Zone Application,” Soil Sci Soc Am 3, 62:5-12 (1998), which are hereby incorporated by reference in their entirety). This produced a barrier effect wherein a hydrophilic barrier was created by the positively charged HDTMA heads, therefore making the organophilic sites on the clay less accessible to DEET. In addition to the interaction of DEET with the hydrophobic HDTMA tails, DEET was likely to interact with the mineral surfaces on the clay (Guangyao et al., “Mechanism(s) Controlling Sorption of Neutral Organic Contaminants by Surfactant Derived and Natural Organic Matter,” Environmental Science & Technology, 30:1553-1557 (1996), which is hereby incorporated by reference in its entirety). Over saturation with HDTMA would not favor this interaction either since there would be less of the exposed mineral surfaces. The reduced binding capacity of 0.5×HDMTA clay for DEET at lower concentrations of DEET was consistent with reports that at this CEC, very little of the surfactant is bound to the external surfaces (Xu et al., “Alternative Model for Cationic Surfactant Absorption by Layer Silicates,” Environ. Sci. Technol., 29:3022-3028 (1995), which is hereby incorporated by reference in its entirety). In line with this theory and the fact that at lower concentrations of DEET, 3×HDMTA clay sorbs more DEET than 0.5× , sorption at lower concentrations of DEET may significantly involve binding of DEET to the HDTMA on the external surfaces.

The complex interaction of DEET with HDTMA clay as outlined above will affect the way DEET interacts on the body, and as a result its bioavailability through the skin will differ from that of pure DEET or DEET in an alcohol solvent. Since one of the major electrolytes in sweat is sodium chloride, leaching into normal saline was used to characterize the release profile of the system once administered in an environment akin to sweat. This release seemed to be driven by a number of processes. The diffusion of DEET out of the carrier was concentration dependent. If the carrier was dispersed in a smaller volume, DEET partitioned mainly in the carrier phase, whereas at higher volumes, it partitioned in the liquid phase, but would not release the total load of drug even at infinite dilution. In addition, the carrier was somewhat degraded upon exposure to normal saline as evidenced by the foaming observed with these samples. HDTMA is a detergent and will foam upon shaking. Sodium chloride seemed to cause desorption of some HDTMA from the clay, resulting in some degree of disintegration, or dissolution of the carrier. Desorption was more significant for HDTMA adsorbed via hydrophobic bonding than via ion exchange (Guangyao et al., “Enhanced Sorption of Organic Contaminants by Smectite Soils Modified with Cationic Surfactant,” Journal of Environmental Quality, 27:806-814 (1998), which is hereby incorporated by reference in its entirety). Whatever DEET was bound to the desorbed HDTMA would likely be released out of the carrier with the HDTMA. HDTMA being a surfactant may also increase the solubility of DEET in normal saline.

Using IPA as a leaching solvent did not seem to change the structural integrity of the HDTMA/clay/DEET matrix. Diluting with the initial 10 ml of IPA resulted in a greater release of DEET followed by a reduced, more linear release profile with subsequent dilutions of 10 ml. The initial release may be the DEET that is weakly bound—most likely the molecules loosely associated with the clay/HDTMA surfaces. The second phase which was linear is most likely to be concentration dependent and can be thought of as perturbation of a partition equilibrium, whereby on dilution, the DEET is thought to be diffusing out of the matrix until the partition equilibrium is re-established. Unlike in the case of normal saline, at infinite dilution, considerably more DEET was available for release from the carrier. Ultimately, nearly all of the DEET (90-97%) could be extracted using IPA. This seemed to also depend on the amount of DEET used. The kinetics of release of DEET from the matrix were not determined only by the drug-carrier interaction, but were also influenced by the DEET in the surrounding medium.

Data from the saline studies suggest that on the skin, the concentration of DEET is likely to remain low since much less is liberated into saline than to solvents like IPA in which DEET is usually formulated. Therefore, as long as there is some degree of binding to clay there will be less DEET available for absorption through the skin.

Although the invention has been described in detail, for the purpose of illustration, it is understood that such detail is for that purpose and variations can be made therein by those skilled in the art without departing from the spirit and scope of the invention which is defined by the following claims.

What is claimed:

1. A complex comprising:
colloid particles having an ion exchange capacity and having attached one or more ligands with a charge and a hydrophobic domain, and
one or more pharmaceutical compounds associated with either or both of the colloid particles and the one or more ligands.

2. The complex according to claim 1, wherein the colloid particles are selected from the group consisting of natural clays, synthetic clays, zeolites, hydrotalcite, kaolinite, talc, halloysite, sepiolite, illite, chlorite, and palygorskite.

3. The complex according to claim 2, wherein the colloid particles are synthetic clay selected from the group consisting of layered hydrous magnesium silicate, synthetic mica-montmorillonite, and mixtures thereof.

4. The complex according to claim 2, wherein the colloid particles are natural clay selected from the group consisting of hectorite, aliettite, beidellite, nontronite, saponite, sauconite, stevensite, swinefordite, volkonskoite, yakhontovite, montmorillonite, bentonite, and mixtures thereof.

5. The complex according to claim 1, wherein the ligand is selected from the group consisting of quaternary ammonium compounds, ionic polymers, ionic surfactants, and mixtures thereof.

6. The complex according to claim 5, wherein the ligand is a quaternary ammonium compound selected from the group consisting of hexadecyltrimethyl ammonium bromide, trimethylphenyl ammonium chloride, and mixtures thereof.

7. The complex according to claim 1, wherein the one or more ligands comprise at least two ligands.

8. The complex according to claim 7, wherein the at least two ligands are different.

9. The complex according to claim 1, wherein the quantity of ligand attached to the colloid particles is in excess of and up to 200% of the ion exchange capacity of the colloid particles.

10. The complex according to claim 1, wherein the pharmaceutical compound is an insect repellent, a sunscreen, or combinations thereof.

11. A method for preventing or reducing surface absorption of one or more pharmaceutical compounds comprising:
applying to a surface a complex comprising:
colloid particles having an ion exchange capacity and having attached one or more igands with a charge and a hydrophobic domain, and
one or more pharmaceutical compounds associated with either or both of the colloid particles and the one or more ligands.

12. The method according to claim 11, wherein the colloid particles are selected from the group consisting of natural clays, synthetic clays, zeolites, hydrotalcite, kaolinite, talc, halloysite, sepiolite, illite, chlorite, and palygorskite.

13. The method according to claim 12, wherein the colloid particles are synthetic clay selected from the group consisting of layered hydrous magnesium silicate, synthetic mica-montmorillonite, and mixtures thereof.

14. The method according to claim 12, wherein the colloid particles are natural clay selected from the group consisting of hectorite, aliettite, beidellite, nontronite, saponite, sauconite, stevensite, swinefordite, volkonskoite, yakhontovite, montmorillonite, bentonite, and mixtures thereof.

15. The method according to claim 11, wherein the ligand is selected from the group consisting of quaternary ammonium compounds, ionic polymers, ionic surfactants, and mixtures thereof.

16. The method according to claim 15, wherein the ligand is a quaternary ammonium compound selected from the group consisting of hexadecyltrimethyl ammonium bromide, trimethylphenyl ammonium chloride, and mixtures thereof.

17. The method according to claim 11, wherein the one or more ligands comprise at least two ligands.

18. The method according to claim 17, wherein the at least two ligands are different.

19. The method according to claim 11, wherein the quantity of ligand attached to the colloid particles is in excess of and up to 200% of the ion exchange capacity of the colloid particles.

20. The method according to claim 11, wherein the one or more pharmaceutical compounds are an insect repellent, a sunscreen, or combinations thereof.

21. The method according to claim 11, wherein the surface is selected from the group consisting of skin and cloth.

22. A method for delayed percutaneous delivery of one or more pharmaceutical compounds comprising:
applying topically a complex comprising:
colloid particles having an ion exchange capacity and having attached one or more ligands with a charge and a hydrophobic domain, and
one or more pharmaceutical compounds associated with either or both of the colloid particles and the one or more ligands.

23. The method according to claim 22, wherein the colloid particles are selected from the group consisting of natural clays, synthetic clays, zeolites, hydrotalcite, kaolinite, talc, halloysite, sepiolite, illite, chlorite, and palygorskite.

24. The method according to claim 23, wherein the colloid particles are synthetic clay selected from the group consisting of layered hydrous magnesium silicate, synthetic mica-montmorillonite, and mixtures thereof.

25. The method according to claim 23, wherein the colloid particles are natural clay selected from the group consisting of hectorite, aliettite, beidellite, nontronite, saponite, sauconite, stevensite, swinefordite, volkonskoite, yakhontovite, montmorillonite, bentonite, and mixtures thereof.

26. The method according to claim 22, wherein the ligand is selected from the group consisting of quaternary ammonium compounds, ionic polymers, ionic surfactants, and mixtures thereof.

27. The method according to claim 26, wherein the ligand is a quaternary ammonium compound selected from the group consisting of hexadecyltrimethyl ammonium bromide, trimethylphenyl ammonium chloride, and mixtures thereof.

28. The method according to claim 22, wherein the one or more ligands comprise at least two ligands.

29. The method according to claim 28, wherein the at least two ligands are different.

30. The method according to claim 22, wherein the quantity of ligand attached to the colloid particles is in excess of and up to 200% of the ion exchange capacity of the colloid particles.

31. The method according to claim 22, wherein the one or more pharmaceutical compounds are an insect repellent, a sunscreen, or combinations thereof.

32. The method according to claim 22, wherein applying topically comprises applying in the form of a solution, lotion, cream, gel, aerosol, impregnated towellette, or pump spray.

33. A surface coated with colloid particles having attached one or more ligands with a charge and a hydrophobic domain and one or more pharmaceutical compounds associated with either or both of the colloid particles and the one or more ligands.

34. The surface according to claim 33, wherein the colloid particles are selected from the group consisting of natural clays, synthetic clays, zeolites, hydrotalcite, kaolinite, talc, halloysite, sepiolite, illite, chlorite, and palygorskite.

35. The surface according to claim 34, wherein the colloid particles are synthetic clay selected from the group consisting of layered hydrous magnesium silicate, synthetic mica-montmorillonite, and mixtures thereof.

36. The surface according to claim 34, wherein the colloid particles are natural clay selected from the group consisting of hectorite, aliettite, beidellite, nontronite, saponite, sauconite, stevensite, swinefordite, volkonskoite, yakhontovite, montmorillonite, bentonite, and mixtures thereof.

37. The surface according to claim 33, wherein the ligand is selected from the group consisting of quaternary ammonium compounds, ionic polymers, ionic surfactants, and mixtures thereof.

38. The surface according to claim 37, wherein the ligand is a quaternary ammonium compound selected from the group consisting of hexadecyltrimethyl ammonium bromide, trimethylphenyl ammonium chloride, and mixtures thereof.

39. The surface according to claim 33, wherein the one or more ligands comprise at least two ligands.

40. The surface according to claim 39, wherein the at least two ligands are different.

41. The surface according to claim 33, wherein the quantity of ligand attached to the colloid particles is in excess of and up to 200% of the ion exchange capacity of the colloid particles.

42. The surface according to claim 33, wherein the one or more pharmaceutical compounds are an insect repellent, a sunscreen, or combinations thereof.

43. The surface according to claim 33, wherein the surface is selected from the group consisting of skin and cloth.

44. A method of making a complex comprising:
providing colloid particles having an ion exchange capacity and having attached one or more ligands with a charge and a hydrophobic domain, and
exposing the colloid particles to one or more pharmaceutical compounds under conditions effective to produce a complex including the one or more pharmaceutical compounds associated with either or both of the colloid particles and the one or more ligands.

45. The method according to claim 44, wherein the colloid particles are selected from the group consisting of natural clays, synthetic clays, zeolites, hydrotalcite, kaolinite, talc, halloysite, sepiolite, illite, chlorite, and palygorskite.

46. The method according to claim 45, wherein the colloid particles are synthetic clay selected from the group consisting of layered hydrous magnesium silicate, synthetic mica-montmorillonite, and mixtures thereof.

47. The method according to claim 45, wherein the colloid particles are natural clay selected from the group consisting of hectorite, aliettite, beidellite, nontronite, saponite, sauconite, stevensite, swinefordite, volkonskoite, yakhontovite, montmorillonite, bentonite, and mixtures thereof.

48. The method according to claim 44, wherein the ligand is selected from the group consisting of quaternary ammonium compounds, ionic polymers, ionic surfactants, and mixtures thereof.

49. The method according to claim 48, wherein the ligand is a quaternary ammonium compound selected from the group consisting of hexadecyltrimethyl ammonium bromide, trimethylphenyl ammonium chloride, and mixtures thereof.

50. The method according to claim 44, wherein the one or more ligands comprise at least two ligands.

51. The method according to claim 50, wherein the at least two ligands are different.

52. The method according to claim 44, wherein the quantity of ligand attached to the colloid particles is in excess of and up to 200% of the ion exchange capacity of the colloid particles.

53. The method according to claim 44, wherein the pharmaceutical compound is an insect repellent, a sunscreen, or combinations thereof.

54. The method according to claim 44, wherein exposing comprises mixing the colloid particles having attached one or more ligands with a solution of the one or more pharmaceutical compounds.

55. The method according to claim 44, wherein the exposing is from about 1 day to about 30 days.

56. The method according to claim 55, wherein the exposing is from about 8 days to about 30 days.
